SEQUENCE LISTING (1 sequence)

SEQ ID NO: 1 - DNA, 1995 bp, Artificial Sequence
  Description of Artificial Sequence Synthetic polynucleotide
atgagaccccagatcctgctgctcctggccctgctgaccctaggcctggcgactacaaggacgatgacgacaagggttcaggcagtggttccgggtcagggggaggtaccatgaactatccgctgactctggaaatggatctggaaaatctcgaagatctcttctgggaactggaccggttggataactacaatgacacaagtctcgtcgagaaccacctgtgtccagctaccgaagggcctttgatggcctcttttaaggctgtgtttgtgcctgtagcctatagcctcattttcctcctcggagttattggaaatgtactcgtgttggtaatccttgagagacaccggcaaacaaggagctcaactgaaaccttcctcttccatctggctgtcgcggatctcctcctcgtgtttatccttccattcgcagttgcggagggttcagtgggatgggtgctcggaacattcttgtgtaagactgtgattgcactccataaggtcaatttctactgctccagtttgctgctcgcctgcatcgctgttgacaggtatctcgccatcgtacatgccgtgcacgcatatcgacacagaagactgctgtccatccatattacctgtggcacaatttggctggtgggattcctgctggcactgcccgagatcctgttcgccaaggtcagtcagggacatcacaataactccctcccacgctgcactttcagtcaagagaatcaggcagaaacccacgcgtggtttacgtctcgattcctttaccatgtagcagggtttctcttgcccatgctggttatgggatggtgctacgttggagtagttcacaggctgcggcaagctcaacgaagaccgcagcggcaaaaagccgtcagagtggctatccttgtcacttccatcttctttctgtgctggagtccttatcacattgtgatattcctggacacactggccaggctgaaagccgtcgataacacatgcaagctcaatggatccctgcctgttgctatcacaatgtgcgaatttctgggtcttgcccattgctgtctgaatcctatgttgtataccttcgctggcgtgaaattccggagtgacctctcaagattgcttactaagttgggctgtacaggccccgcttctctgtgtcagttgtttccttcatggcgacggtccagccttagtgaatctgagaacgctactagcctcaccactttcggaagcggttctggcagtgggagtatgcatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagctttgtgcacgacgatgat

(19) United States
(12) Patent Application Publication
(10) Pub. No.: US 2022/0186209 A1
(43) Pub. Date: Jun. 16, 2022

(54) METHODS OF ANTIBODY PANNING AGAINST TARGET PROTEINS

(71) Applicant: Charles River Laboratories, Inc., Wilmington, MA (US)

(72) Inventors: Jacob Glanville, San Francisco, CA (US); Sawsan Youssef, Menlo Park, CA (US); Valerie Chiou, South San Francisco, CA (US); David Maurer, South San Francisco, CA (US)

(21) Appl. No.: 17/685,881

(22) Filed: Mar. 3, 2022

Related U.S. Application Data
(63) Continuation of application No. PCT/US2020/050567, filed on Sep. 11, 2020.
(60) Provisional application No. 62/900,321, filed on Sep. 13, 2019.

(51) Int. Cl. C12N 15/10

(52) U.S. Cl. CPC C12N 15/1058

(57) ABSTRACT

Disclosed herein are methods for selecting polypeptides that specifically bind to a target protein. In some cases, the methods involve panning a library of polypeptides for target polypeptides that bind to a target protein on the surface of a cell. Additionally, the disclosure provides cell lines expressing target proteins for use with the disclosed methods.

### CROSS-REFERENCE

This application is a continuation application of International Application No. PCT/US2020/050567, filed Sep. 11, 2020, which claims the benefit of U.S. Provisional Application No. 62/900,321, filed Sep. 13, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 2, 2022, is named 60864-703_301_SL.txt and is 4,096 bytes in size.

### BACKGROUND

Membrane-bound proteins, such as G protein-coupled receptors (GPCRs), offer attractive targets for monoclonal antibodies (mAbs). However, for various reasons, it may be challenging to develop antibodies that target GPCRs and other proteins. Cellular-based panning methods may allow membrane-bound proteins to be screened in their natural conformation against different antibodies. Provided herein are methods for selecting polypeptides that bind to target proteins, including GPCRs and other challenging target proteins. Further provided herein are engineered cell lines that express target proteins that are suitable for use with the methods provided herein.

### SUMMARY

In one aspect, a method is provided for selecting for a polypeptide that selectively binds to a target protein, the method comprising: (a) contacting a first polypeptide pool comprising a plurality of polypeptides with a first entity that does not comprise the target protein to form a first mixture; (b) removing the first entity from the first mixture, thereby generating a first depleted polypeptide pool; (c) contacting the first depleted polypeptide pool with a second entity that comprises the target protein at its surface; (d) collecting polypeptides that bind to the second entity, thereby generating a target polypeptide pool; (e) contacting a second polypeptide pool comprising a plurality of polypeptides with the first entity to form a second mixture; (f) removing the first entity from the second mixture, thereby generating a second depleted polypeptide pool; (g) contacting the second depleted polypeptide pool with a third entity that does not comprise the target protein, wherein the third entity is the same or different from the first entity; (h) collecting polypeptides that bind to the third entity, thereby generating an off-target polypeptide pool; and (i) identifying at least one polypeptide that is present in the target polypeptide pool and is not present in the off-target polypeptide pool, thereby selecting for a polypeptide that selectively binds to the target protein. In some cases, the method further comprises, prior to (i), performing one or more rounds of (a)-(h), each successive round using a target polypeptide pool generated in (d) as a first polypeptide pool in (a), and using an off-target polypeptide pool generated in (h) as a second polypeptide pool in (e). In some cases, the identifying of (i) comprises sequencing a polynucleotide tag attached to the at least one polypeptide. In some cases, the target polypeptide pool comprises a plurality of target polypeptide, and the off-target polypeptide pool comprises a plurality of off-target polypeptides. In some cases, the identifying of (i) comprises sequencing polynucleotide tags attached to the plurality of target polypeptides and the plurality of off-target polypeptides. In some cases, each of the polynucleotide tags is different for each target polypeptide and for each off-target polypeptide. In some cases, the first entity, the second entity, and the third entity are of a same type. In some cases, for each successive round of (a)-(h), the first entity, the second entity, and the third entity are of a different type from a preceding round. In some cases, for each successive round of (a)-(h), the first entity, the second entity, and the third entity are from a different species than from a preceding round. In some cases, the first entity comprises a plurality of first entities, the second entity comprises a plurality of second entities, and the third entity comprises a plurality of third entities. In some cases, for each successive round of (a)-(h), decreasing a number of the plurality of second entities contacted with the first depleted polypeptide pool, and decreasing a number of the plurality of third entities contacted with the second depleted polypeptide pool, as compared to a preceding round. In some cases, for each successive round of (a)-(h), a number of first entities is the same as a preceding round. In some cases, the method further comprises performing one or more wash steps on the second entity and the third entity. In some cases, the method further comprises, for each successive round of (a)-(h), increasing a number of wash steps from a preceding round. In some cases, the target protein is a cell-surface protein, a membrane-bound protein, or a protein engineered to be expressed at a cell surface. In some cases, the target protein is a transmembrane protein or an integral membrane protein. In some cases, the transmembrane protein is a single-pass transmembrane protein or a multi-pass transmembrane protein. In some cases, the target protein is selected from the group consisting of: a ligand-gated ion channel, a voltage-gated ion channel, and a G protein-coupled receptor (GPCR). In some cases, the second entity is genetically engineered to express the target protein. In some cases, the second entity is genetically engineered to express the target protein at a cell surface. In some cases, the second entity is genetically engineered to stably express the target protein. In some cases, the second entity is genetically engineered to transiently express the target protein. In some cases, the target protein comprises a detectable label. In some cases, the detectable label is a fluorescent label. In some cases, the fluorescent label is a fluorescent protein. In some cases, the target protein comprises a barcode. In some cases, the barcode is a polynucleotide tag. In some cases, the target protein comprises a sequence that localizes the target protein to a cell surface, that prevents the target protein from being internalized from a cell surface, does not comprise a sequence that causes the target protein to be internalized upon ligand binding, or any combination thereof. In some cases, the target protein is expressed in a natural conformation state. In some cases, any one of the first entity, the second entity, and the third entity is a cell sample, a cell lysate sample, or a cell fragment sample. In some cases, any one of the first entity, the second entity, and the third entity is a cell membrane fraction. In some cases, any one of the first entity, the second entity, and the third entity is a polyliposome. In some cases, any one of the first entity, the second entity, and the third entity comprises a whole cell. In some cases, any one of the first entity, the second entity, and the third entity comprises an adherent cell. In some cases, any one of the first entity, the second entity, and the third entity comprises a suspension cell. In some cases, the first polypeptide pool, the second polypeptide pool, or both, is an antibody library comprising a plurality of antibodies or antibody fragments. In some cases, the first polypeptide pool, the second polypeptide pool, or both, comprises at least two polypeptides. In some cases, the first polypeptide pool, the second polypeptide pool, or both comprises at least ten polypeptides. In some cases, the first polypeptide pool, the second polypeptide pool, or both, comprises at least 100 polypeptides. In some cases, the first polypeptide pool, the second polypeptide pool, or both, comprises at least 1,000 polypeptides. In some cases, the first polypeptide pool, the second polypeptide pool, or both, comprises at least 10,000 polypeptides. In some cases, the first polypeptide pool, the second polypeptide pool, or both, comprises at least 100,000 polypeptides. In some cases, the first polypeptide pool, the second polypeptide pool, or both, comprises at least 1,000,000 polypeptides. In some cases, the method further comprises sorting the second entity, the third entity, or both based on a detectable label expressed thereon. In some cases, the method further comprises screening the at least one polypeptide for binding to the target protein. In some cases, the screening comprises performing a functional assay. In some cases, the method further comprises performing one or more affinity maturation steps on the at least one polypeptide to generate a polypeptide with increased affinity for the target protein. In some cases, the first entity, the third entity, or both, are genetically engineered to have a knock-out or a knock-down of the target protein. In some cases, the target polypeptide pool comprises a plurality of target polypeptides. In some cases, each of the plurality of target polypeptides has a dissociation constant (KD) of less than about 100 nM for the target protein. In some cases, each of the plurality of target polypeptides has a KD of less than about 10 nM for the target protein. In some cases, each of the plurality of target polypeptides has a KD of less than about 1 nM for the target protein.

In another aspect, a target polypeptide identified by any of the preceding methods is provided herein. In some cases, the target polypeptide has a KD of less than 100 nM for the target protein. In some cases, the target polypeptide has a KD of less than about 10 nM for the target protein. In some cases, the target polypeptide has a KD of less than about 1 nM for the target protein.

In another aspect, a cell or cellular sample is provided for use in any of the preceding methods, wherein the cell or cellular sample is genetically modified to express a target protein. In some cases, the target protein comprises a signal tag that localizes the target protein to a cell surface. In some cases, the target protein comprises a signal tag that prevents or reduces internalization of the target protein from a cell surface, does not comprise a sequence that causes the target protein to be internalized upon ligand binding, or both. In some cases, the target protein comprises a detectable label. In some cases, the label comprises a fluorescent protein. In some cases, the target protein comprises a FLAG tag. In some cases, the target protein comprises a polynucleotide tag. In some cases, the target protein is a cell-surface protein, a membrane-bound protein, or a protein engineered to be expressed at a cell surface. In some cases, the protein is a transmembrane protein or an integral membrane protein. In some cases, the target protein is a single-pass transmembrane protein or a multi-pass transmembrane protein. In some cases, the target protein is selected from the group consisting of: a ligand-gated ion channel, a voltage-gated ion channel, and a G protein coupled receptor (GPCR). In some cases, the cell or cellular sample stably expresses the target protein. In some cases, the cell or cellular sample transiently expresses the target protein. In some cases, the cell or cellular sample comprises a plurality of cells, each of the plurality of cells expressing the target protein at a high copy number. In some cases, the cell or cellular sample is a cell sample, a cell lysate sample, or a cell fragment sample. In some cases, the cell or cellular sample is a cell membrane fraction. In some cases, the cell or cellular sample is a whole cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a non-limiting example of a bioengineered G protein-coupled receptor (GPCR) construct suitable for use with the methods disclosed herein.

FIG. 1B depicts a non-limiting example of a vector suitable for stable transfection of host cell lines in accordance with embodiments of the disclosure.

FIG. 1C depicts a non-limiting example of a bioengineered GPCR construct suitable for use with the methods disclosed herein.

FIG. 1D depicts a non-limiting example of a vector suitable for lentiviral transduction of host cell lines in accordance with embodiments of the disclosure.

FIG. 1E depicts a non-limiting example of a mammalian vector modified with a GPCR construct in accordance with embodiments of the disclosure.

FIG. 1F depicts a non-limiting example of a lentiviral vector modified with a GPCR construct in accordance with embodiments of the disclosure.

FIGS. 2A-2C depict a non-limiting example of FACS plots (FIGS. 2B, 2C) demonstrating high co-expression of CXCR5 and GFP on the cell surface of mammalian cells stably expressing a GPCR construct (FIG. 2A) of the disclosure.

FIG. 2D depicts a non-limiting example of fluorescent microscopy data of mammalian cells stably expressing a GPCR construct of the disclosure, demonstrating translocation of GFP to the cell surface.

FIGS. 2E and 2F depict a non-limiting example of FACS plots (FIG. 2F) demonstrating high co-expression of CXCR5 and GFP on the cell surface of mammalian cells transduced with a lentiviral vector of the disclosure (and stably expressing a GPCR construct (FIG. 2E) of the disclosure).

FIG. 2G depicts a non-limiting example of fluorescent microscopy data of mammalian cells transduced with a lentiviral vector of the disclosure, demonstrating translocation of GFP to the cell surface.

FIG. 3A depicts a non-limiting example of a cell panning methodology in accordance with embodiments of the disclosure.

FIG. 3B depicts a non-limiting example of a cell panning methodology in accordance with embodiments of the disclosure.

FIG. 4A and FIG. 4B depict non-limiting examples of cell panning methodologies in accordance with embodiments of the disclosure.

FIG. 5 depicts a non-limiting example of an affinity maturation methodology in accordance with embodiments of the disclosure.

FIG. 6A and FIG. 6B depict non-limiting examples of methods of extracting VH and VK from cell panning output pools in accordance with embodiments of the disclosure.

FIG. 6C depicts non-limiting examples of log-log frequency plots generated from next generation sequencing results of clones in accordance with embodiments of the disclosure.

FIG. 6D depicts a non-limiting example of data showing the distribution of non-selected clones and selected clones in accordance with embodiments of the disclosure.

FIG. 6E depicts a non-limiting example of the statistical properties of anti-CXCR5 antibodies selected according to methods provided herein.

FIG. 6F depicts a non-limiting example of the humanness of anti-CXCR5 antibodies selected in accordance with the methods provided herein.

FIG. 6G depicts non-limiting examples of FACS plots demonstrating CXCR5-binding scFvs selected in accordance with the methods provided herein.

FIG. 6H depicts non-limiting examples of FACS plots demonstrating binding of antibodies to CXCR5 in accordance with the methods provided herein.

FIGS. 7A-7D depict non-limiting examples of data generated from assays testing the functionality of CXCR5 antibodies selected in accordance with the methods provided herein.

FIG. 8 depicts non-limiting examples of FACS plots demonstrating binding of affinity matured antibodies to CXCR5 in accordance with the methods provided herein.

### DETAILED DESCRIPTION

Provided herein are methods for selecting polypeptides that selectively bind to a target protein. Generally, the methods provided herein involve the use of cell panning methods that allow the target protein to be expressed in its native conformation and natural environment. The methods generally involve the use of multiple rounds of cell panning to select for polypeptides that bind with high affinity to the target polypeptide. The methods provided herein may be suitable for developing polypeptides that bind to difficult or challenging target proteins (e.g., those target proteins for which it is generally difficult or challenging to design polypeptides that selectively bind thereto), such as G protein-coupled receptors (GPCRs). The disclosure further provides target proteins that are suitable for use with the cell panning methods provided herein, as well as cell lines engineered to express said target proteins.

Methods of Selecting Polypeptides that Selectively Bind to a Target Protein

Disclosed herein are methods for selecting polypeptides that selectively bind to a target protein. In one aspect, a method comprises: (a) contacting a first polypeptide pool comprising a plurality of polypeptides with a first entity that does not comprise the target protein to form a first mixture; (b) removing the first entity from the first mixture, thereby generating a first depleted polypeptide pool; (c) contacting the first depleted polypeptide pool with a second entity that comprises the target protein at its surface; (d) collecting polypeptides that bind to the second entity, thereby generating a target polypeptide pool; (e) contacting a second polypeptide pool comprising a plurality of polypeptides with the first entity to form a second mixture; (f) removing the first entity from the second mixture, thereby generating a second depleted polypeptide pool; (g) contacting the second depleted polypeptide pool with a third entity that does not comprise the target protein, wherein the third entity is the same or different from the first entity; (h) collecting polypeptides that bind to the third entity, thereby generating an off-target polypeptide pool; and (i) identifying at least one polypeptide that is present in the target polypeptide pool and is not present in the off-target polypeptide pool, thereby selecting for a polypeptide that selectively binds to the target protein.

In various aspects, the methods provide for one or more first depletion steps. In some cases, a first depletion step involves contacting a first polypeptide pool with a first entity. In some cases, the first entity does not comprise a target protein. In some cases, the first entity does not express a target protein. In some cases, the first polypeptide pool is incubated with the first entity under conditions (e.g., appropriate temperature, time, buffer conditions, etc.) such that one or more polypeptides bind to the surface of the first entity. In some cases, the one or more polypeptides may non-specifically bind to the surface of the first entity and polypeptides that do not bind to the surface of the first entity remain in the solution. In some cases, the polypeptides that do not bind to the surface of the first entity may include one or more target polypeptides that bind to a target protein. Contacting the first polypeptide pool with the first entity may deplete non-specific and/or off-target polypeptides from the first polypeptide pool and may enrich the polypeptide pool for polypeptides that bind to the target protein.

The first polypeptide pool may comprise a plurality of polypeptides. The first polypeptide pool may comprise a plurality of diverse polypeptides. In some cases, the first polypeptide pool may comprise at least two, at least ten, at least 100, at least 1,000, at least 10,000, at least 100,000, at least 1,000,000, at least 10,000,000, at least 100,000,000, or more polypeptides. In some cases, the first polypeptide pool may comprise at least two, at least ten, at least 100, at least 1,000, at least 10,000, at least 100,000, at least 1,000,000, at least 10,000,000, at least 100,000,000, or more diverse polypeptides. In some cases, the first polypeptide pool may be a library of polypeptides. In exemplary examples, the first polypeptide pool is an antibody library. The antibody library may comprise a plurality of antibodies or antibody fragments. In a particular example, the antibody library may be a highly diverse antibody library. In some cases, the antibody library may be a phage antibody library. In some cases, the phage antibody library may be heated prior to each first depletion step. In some cases, the first polypeptide pool may be a target polypeptide pool generated after a single round of panning (e.g., the output from a single round of panning may serve as the input for a subsequent round of panning).

The term polypeptide can be any protein, peptide, protein fragment, or any component thereof. A polypeptide can be a protein naturally occurring in nature or a protein that is ordinarily not found in nature. A polypeptide can consist largely of the standard twenty protein-building amino acids or it can be modified to incorporate non-standard amino acids. A polypeptide can be modified, e.g., adding any number of biochemical functional groups, including phosphorylation, acetylation, acylation, formylation, alkylation, methylation, lipid addition (e.g., palmitoylation, myristoylation, prenylation, etc.), and carbohydrate addition (e.g., N-linked and O-linked glycosylation, etc.). A polypeptide can include an antibody or antibody fragment.

The term “antibody” as used herein refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and portions thereof; including, for example, an immunoglobulin molecule, a polyclonal antibody, a monoclonal antibody, a recombinant antibody, a chimeric antibody, a humanized antibody, a CDR-grafted antibody, F(ab)₂, Fv, scFv, IgGΔCH₂, F(ab′)₂, scFv2CH3, F(ab), VL, VH, scFv4, scFv3, scFv2, dsFv, Fv, scFv-Fc, (scFv)₂, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies).

In various aspects, the first entity may comprise a plurality of first entities. In some cases, the first entity may be a cell (e.g., a biological cell). Non-limiting examples of cells suitable for use with the methods are provided herein. In a particular example, a cell may be a mammalian cell. The cell may be an immortalized cell or a primary cell. In some cases, the cell may be a whole cell or an intact cell. In some cases, the cell may be an adherent cell. In some cases, the cell may be a suspension cell. In some cases, the cell may not be genetically engineered. In some cases, the cell may be genetically engineered to knock-down or knock-out the target protein. In some cases, the first entity may be a cell-like particle. In some cases, the first entity may have a cell membrane, or may have one or more components typically found in a cell membrane (e.g., phospholipids, cholesterol, proteins, carbohydrates, etc.). In some cases, the first entity may be a cellular sample, a cell lysate sample, or a cell fragment sample. In some cases, the first entity may be a cell membrane fraction. In some cases, the first entity may be a polyliposome. In some cases, the first entity may be a cell fraction on a bead. In some cases, the first entity may be a parental cell line from which a genetically engineered cell line expressing the target protein is generated (e.g., a second entity, as described herein).

In various aspects, the methods can involve removing the first entity from the first mixture such that off-target and/or non-specific polypeptides are removed from the first polypeptide pool, thereby generating a first depleted polypeptide pool. In some cases, the first depleted polypeptide pool comprises a plurality of polypeptides comprising at least one polypeptide that specifically binds to the target protein. In some cases, removing may involve separating the first entity (comprising one or more non-specific polypeptides bound thereto) from the solution, thereby removing the non-specific polypeptides bound to the first entity.

In various aspects, the methods further involve performing one or more first enrichment steps. In some cases, a first enrichment step may comprise contacting the first depleted polypeptide pool with a second entity. In some cases, the second entity may comprise the target protein. In some cases, the second entity may express the target protein. In exemplary cases, the second entity comprises the target protein at a cell surface, such that it is capable of being bound by at least one target polypeptide. In some cases, the first depleted polypeptide pool may be incubated with the second entity under conditions in which at least one target polypeptide binds to a target protein. In some cases, the target protein may be expressed at the cell surface such that the target protein is in its native conformation.

In various aspects, the second entity may comprise a plurality of second entities. In some cases, the second entity may be a cell (e.g., a biological cell). Non-limiting examples of cells suitable for use with the methods are provided herein. In a particular example, a cell may be a mammalian cell. In some cases, the cell may be an immortalized cell or a primary cell. In some cases, the cell may be a whole cell or an intact cell. In some cases, the cell may be an adherent cell. In some cases, the cell may be a suspension cell. In some cases, the second entity may be a cell-like particle. In some cases, the second entity may comprise a cell membrane, or one or more components typically found in a cell membrane (e.g., phospholipids, cholesterol, protein, carbohydrates, etc.). In some cases, the second entity may be a cellular sample, a cell lysate sample, or a cell fragment sample. In some cases, the second entity may be a cell membrane fraction. In some cases, the second entity may be a polyliposome. In some cases, the second entity may be a cell fraction on a bead. In some cases, the second entity (e.g., a cell) may be genetically engineered to express the target protein. In some cases, the second entity may be genetically engineered to express the target protein at the cell surface. In some cases, the second entity may be genetically engineered to transiently express the target protein. In some cases, the second entity may be genetically engineered to stably express the target protein. In some cases, the second entity may be derived from a parental cell line that does not comprise the target protein (e.g., the first entity). In some cases, the second entity may be generated by genetically modifying a parental cell line to express the target protein. In some cases, the second entity may be of the same cell type as the first entity. In some cases, the second entity may be from the same species as the first entity. Methods of generating cell lines expressing target proteins suitable for use with the cell panning methods are provided herein.

In various aspects, the methods can further comprise collecting target polypeptides that bind to the target protein on a surface of the second entity, thereby generating a target polypeptide pool. In some cases, the collecting may involve separating the second entity (comprising one or more target polypeptides bound thereto) from the solution. In some cases, the target polypeptide pool may comprise one or more target polypeptide. In some cases, the target polypeptide pool may comprise a plurality of target polypeptides.

In some cases, the collecting may further involve one or more wash steps. In some cases, the second entity may be washed to remove any unbound polypeptides. In some cases, the second entity may be washed to remove any polypeptides that non-specifically bind to the second entity. In some cases, the one or more wash steps may involve washing with a buffered solution. The stringency of the one or more wash steps may be adjusted or altered to increase the stringency or to decrease the stringency of the wash (e.g., by altering the pH, temperature, number of washes, and the like). In some cases, as described in more detail herein, the stringency of the one or more wash steps may be increased in subsequent rounds of panning to select for target polypeptides having increased selectivity for the target protein. Generally, the one or more wash steps may remove unbound polypeptides from the second entity but may not remove target polypeptides selectively bound to the target protein.

In some aspects, the collecting may optionally comprise sorting the second entity based on the expression levels or the amount of the target protein. In some cases, the second entity may be sorted based on the level of a detectable marker attached to the target protein. In some cases, the second entity comprising the highest levels of target protein may be sorted. In some cases, the detectable marker may be a fluorescent protein (e.g., green fluorescent protein (GFP), red fluorescent protein (RFP), or blue fluorescent protein (BFP)). In some cases, the second entity may be sorted by fluorescence-activated cell sorting (FACS); however, any other suitable method may be employed.

In various aspects, the collecting may further comprise eluting one or more target polypeptides bound to the target protein. In some cases, eluting may be performed by incubating the second entity in a high pH solution (e.g., triethylamine solution), followed by neutralization with a neutral pH solution. Elution may remove the one or more target polypeptides bound to the target protein into the solution (e.g., release the one or more target polypeptides into the solution). Thus, the solution, after the elution step, may comprise the one or more target polypeptides. Optionally, after elution, the second entity may be shredded (e.g., by passing the second entity through a QlAshredder).

In various aspects, the methods provide for one or more second depletion steps. In some cases, a second depletion step may involve contacting a second polypeptide pool with the first entity. In some cases, the first entity does not comprise a target protein. In some cases, the first entity does not express a target protein. In some cases, the second polypeptide pool may be incubated with the first entity under conditions (e.g., appropriate temperature, time, buffer conditions, etc.) such that one or more polypeptides bind to the surface of the first entity. In some cases, the one or more polypeptides may non-specifically bind to the surface of the first entity and polypeptides that do not bind to the surface of the first entity remain in the solution. In some cases, the polypeptides that do not bind to the surface of the first entity may include one or more target polypeptides that bind to a target protein. Contacting the second polypeptide pool with the first entity may deplete non-specific and/or off-target polypeptides from the second polypeptide pool, and may enrich the polypeptide pool for polypeptides that bind to the target protein.

The second polypeptide pool may comprise a plurality of polypeptides. The second polypeptide pool may comprise a plurality of diverse polypeptides. In some cases, the second polypeptide pool may comprise at least two, at least ten, at least 100, at least 1,000, at least 10,000, at least 100,000, at least 1,000,000, at least 10,000,000, at least 100,000,000, or more polypeptides. In some cases, the second polypeptide pool may comprise at least two, at least ten, at least 100, at least 1,000, at least 10,000, at least 100,000, at least 1,000,000, at least 10,000,000, at least 100,000,000, or more diverse polypeptides. In some cases, the second polypeptide pool may be a library of polypeptides. In exemplary examples, the second polypeptide pool is an antibody library. The antibody library may comprise a plurality of antibodies or antibody fragments. In a particular example, the antibody library may be a highly diverse antibody library. In some cases, the antibody library may be a phage antibody library. In some cases, the phage antibody library may be heated prior to each second depletion step. In some cases, the second polypeptide pool may be an off-target polypeptide pool generated after a single round of panning (e.g., the output from a single round of panning may serve as the input for a subsequent round of panning).

In various aspects, the methods can involve removing the first entity from the second mixture such that off-target and/or non-specific polypeptides are removed from the second polypeptide pool, thereby generating a second depleted polypeptide pool. In some cases, removing may involve separating the first entity (comprising one or more non-specific polypeptides bound thereto) from the second mixture, thereby removing the non-specific polypeptides bound to the first entity.

In various aspects, the methods provided herein further involve performing one or more second enrichment steps. In some cases, the one or more second enrichment steps may involve contacting the second depleted polypeptide pool with a third entity. In some cases, the third entity does not comprise the target protein. In some cases, the third entity does not express the target protein. In some cases, the second depleted polypeptide pool may be incubated with the third entity under conditions such that at least one polypeptide binds to the third entity. In some cases, one or more polypeptides may non-specifically bind to the third entity.

Generally, the one or more second enrichment steps may be performed in parallel with the first enrichment steps described above. Put another way, the first depleted polypeptide pool may be used in a first enrichment step to select for target polypeptides that selectively bind to the target protein, and the second depleted polypeptide pool may be used in a second enrichment step to select for off-target polypeptides that non-specifically bind to the third entity. The pools generated from the first enrichment and second enrichment steps may then be compared to identify target polypeptides that selectively bind to the target protein and do not bind to the third entity.

In various aspects, the third entity may comprise a plurality of third entities. In some cases, the third entity may be a cell (e.g., a biological cell). Non-limiting examples of cells suitable for use with the methods are provided herein. In a particular example, a cell may be a mammalian cell. The cell may be an immortalized cell or a primary cell. In some cases, the cell may be a whole cell or an intact cell. In some cases, the cell may be an adherent cell. In some cases, the cell may be a suspension cell. In some cases, the cell may not be genetically engineered. In some cases, the cell may be genetically engineered to knock-down or knock-out the target protein. In some cases, the third entity may be a cell-like particle. In some cases, the third entity may have a cell membrane, or one or more components typically found in a cell membrane (e.g., phospholipid, cholesterol, protein, carbohydrates, etc.). In some cases, the third entity may be a cellular sample, a cell lysate sample, or a cell fragment sample. In some cases, the third entity may be a cell membrane fraction. In some cases, the third entity may be a polyliposome. In some cases, the third entity may be a cell fraction on a bead. In some cases, the third entity may be a parental cell line from which a genetically engineered cell line expressing the target protein is generated (e.g., a second entity, as described herein). In some cases, the third entity and the first entity are the same. In some cases, the third entity and the first entity are different.

In various aspects, the methods further comprise collecting one or more polypeptides that bind to the third entity, thereby generating an off-target polypeptide pool. In some cases, the collecting may involve removing the solution containing one or more off-target polypeptides that bind to the third entity. In some cases, the off-target polypeptide pool may comprise one or more off-target polypeptides. In some cases, the off-target polypeptide pool may comprise a plurality of off-target polypeptides.

In some cases, the collecting may further involve one or more wash steps. In some cases, the third entity may be washed to remove any unbound polypeptides. In some cases, the one or more wash steps may involve washing with a buffered solution. The stringency of the one or more wash steps may be adjusted or altered to increase the stringency or to decrease the stringency of the wash (e.g., by altering the pH, temperature, number of washes, and the like).

In various aspects, the methods may further involve identifying at least one polypeptide that is present in the target polypeptide pool and is not present in the off-target polypeptide pool. In some cases, the polypeptides present in the target polypeptide pool and the polypeptides present in the off-target polypeptide pool may be sequenced, the identities of polypeptides present in each pool may be compared, and polypeptides present in the target polypeptide pool but not present in the off-target polypeptide pool may be identified. In some cases, the identifying may involve sequencing a polynucleotide tag (e.g., a polynucleotide barcode) attached to the polypeptides. In some cases, the polynucleotide tags are different for each unique target polypeptide and for each unique off-target polypeptide.

In various aspects, the methods may further involve amplifying the polypeptides present in the target polypeptide pool, and/or amplifying the polypeptides present in the off-target polypeptide pool. After amplification, the amplified target polypeptide pool may again be subjected to one or more rounds of panning. For example, the amplified target polypeptide pool may be subjected to another round of first depletion and first enrichment to further select for target polypeptides having increased selectivity or affinity for the target protein. In parallel, the amplified off-target polypeptide pool may be subjected to one or more rounds of panning. For example, the amplified off-target polypeptide pool may be subjected to another round of second depletion and second enrichment. In some cases, the one or more rounds of panning may be performed prior to identifying the at least one polypeptide present in the target polypeptide pool and not present in the off-target polypeptide pool. In some cases, one, two, three, four, five, or more than five rounds of panning may be performed. In some cases, the target polypeptide pool generated from each round of panning may be used as the first polypeptide pool for another round of panning. Similarly, the off-target polypeptide pool generated from each round of panning may be used as the second polypeptide pool for another round of panning.

In some cases, the first entity, the second entity, and the third entity within a single round of panning are of the same type. In some cases, the first entity, the second entity, and the third entity within a single round of panning may be the same cell type. In some cases, the first entity, the second entity, and the third entity within a single round of panning may be from the same species. By way of illustration only, the first entity, the second entity, and the third entity within a single round of panning may be a Jurkat cell line (human). As described above, the first and third entity generally do not comprise the target protein, whereas the second entity may be genetically engineered to express the target protein.

In a subsequent round of panning, the first entity, the second entity, and the third entity may be of a different type from a preceding round. In some cases, the first entity, the second entity, and the third entity may be of a different cell type from a preceding round. In some cases, the first entity, the second entity, and the third entity may be from a different species from a preceding round. By way of illustration only, the first entity, the second entity, and the third entity in a first round of panning may be a Jurkat cell line (human); the first entity, the second entity, and the third entity in a second round of panning may be a CHO cell line (Chinese hamster).

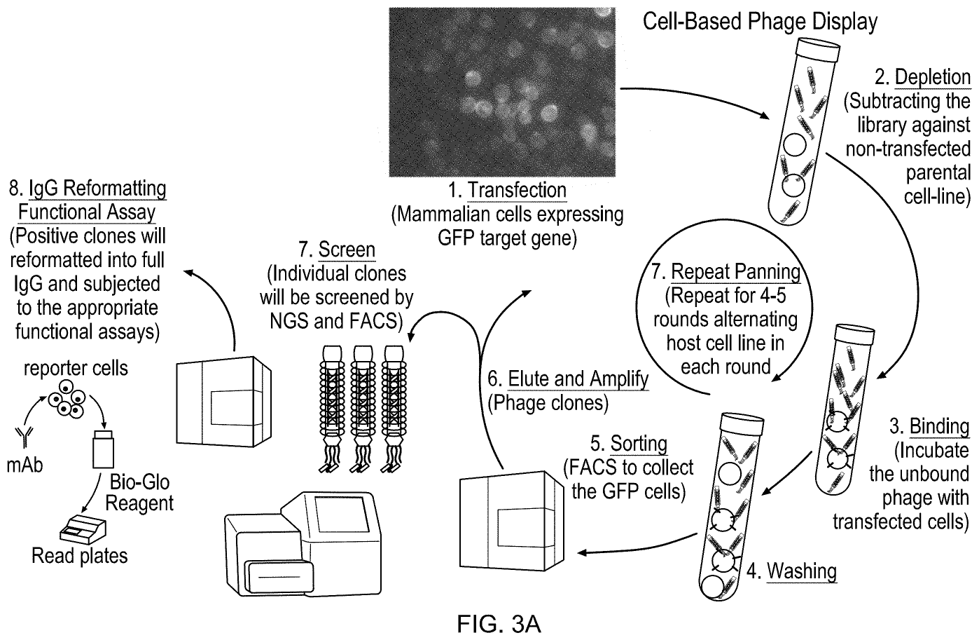

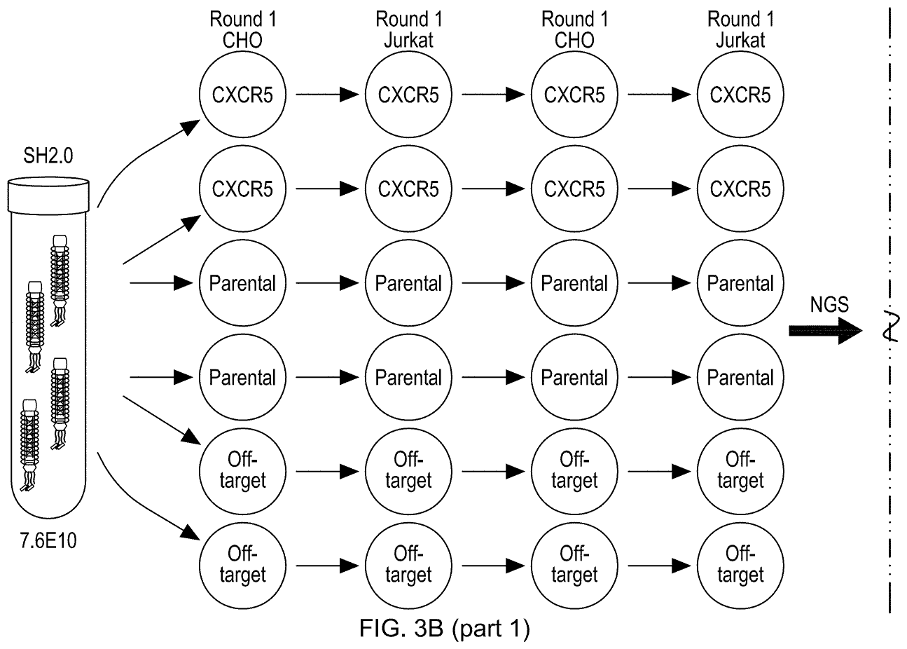

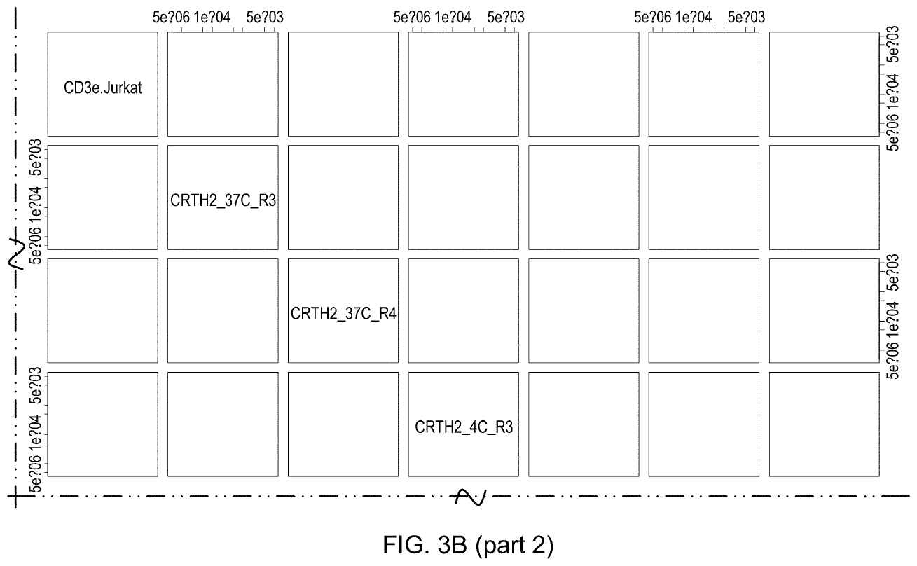

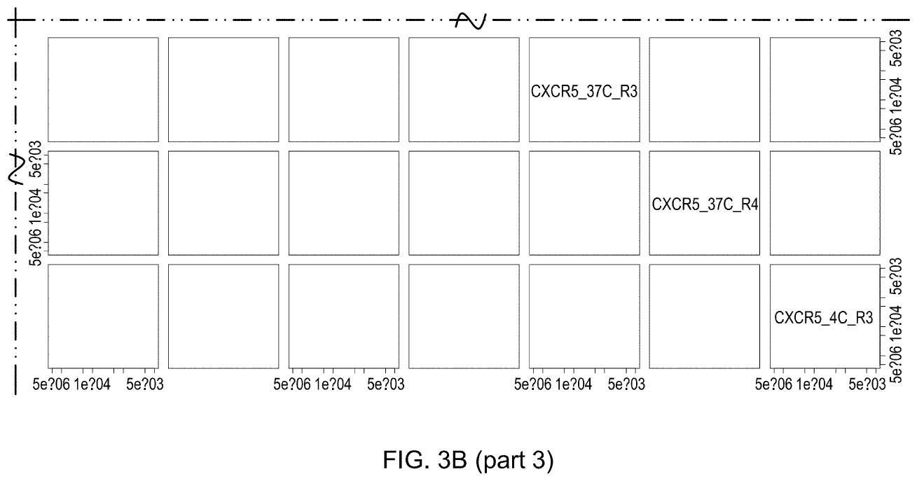

FIG. 3B depicts an example of a cell panning workflow in accordance with embodiments herein. A first round of panning may be performed. The first round of panning may include a first depletion step, a first enrichment step, a second depletion step, and a second enrichment step, as described below. Briefly, a first depletion step may be performed by incubating a first polypeptide pool (e.g., an aliquot of a SuperHuman 2.0 phage antibody library) with a first entity (e.g., a CHO parental cell line) that does not comprise the target protein. The first entity may be removed and the resulting first depleted polypeptide pool may be subjected to a first enrichment step in which the first depleted polypeptide pool may be incubated with a second entity (e.g., an engineered CHO cell line expressing the target polypeptide) such that at least one polypeptide binds to the target protein on the second entity. The second entity may then be collected and then the at least one target polypeptide may be eluted from the second entity, thereby generating a target polypeptide pool. For subsequent rounds of panning, the target polypeptide pool serves as the first polypeptide pool (e.g., the output from the first round of panning (the target polypeptide pool) becomes the input for the second round of panning). Additionally, in parallel with the first depletion step and the first enrichment step described above, a second depletion step may be performed by incubating a second polypeptide pool (e.g., an aliquot of a SuperHuman 2.0 phage antibody library) with the first entity (e.g., a CHO parental cell line) that does not comprise the target protein. The first entity may be removed and the resulting second depleted polypeptide pool may be subjected to a second enrichment step in which the second depleted polypeptide pool may be incubated with a third entity (e.g., a CHO parental cell line) such that at least one polypeptide binds to the third entity. The third entity may then be collected and then the at least one polypeptide may be eluted from the third entity, thereby generating an off-target polypeptide pool. For subsequent rounds of panning, the off-target polypeptide pool serves as the second polypeptide pool (e.g., the output (the off-target polypeptide pool) from the first round of panning becomes the input for the second round of panning). In this example, four rounds of panning are performed with rounds 2, 3, and 4 starting with the output from the previous rounds. In each subsequent round of panning, the cell type is alternated. Additionally or alternatively, in parallel, multiple rounds of panning may be performed on a cell line expressing an off-target protein.

In various aspects, the conditions of the first enrichment step and the second enrichment step may be altered. In one non-limiting example, the number of second entities contacted with the first depleted polypeptide pool, and the number of third entities contacted with the second depleted polypeptide pool may vary in subsequent rounds. In some cases, the number of second entities and the number of third entities used in the first enrichment and second enrichment, respectively, may be decreased in a subsequent round of panning. In some aspects, the number of first entities used in the first depletion step and the second depletion step may be the same for each round of panning.

Additionally or alternatively, the incubation time for the first enrichment step and the second enrichment step may vary in subsequent rounds of panning. In some cases, the incubation time for the first enrichment step and the second enrichment step may be decreased in a subsequent round of panning. In some aspects, the incubation time for the first depletion step and the second depletion step may be the same for each round of panning.

Additionally or alternatively, the one or more wash steps in each round of panning may be varied to increase or decrease stringency of the panning. In a non-limiting example, the number of wash steps may be increased in each subsequent round of panning (e.g., two wash steps in the first round, four wash steps in the second round, etc.). In some cases, the wash steps may be altered in other ways (e.g., wash time, wash temperature, buffer composition, pH, and the like). Generally, the wash steps may be altered to increase the stringency such that the methods select for target polypeptides with high affinity for the target protein.

In some instances, the target polypeptides obtained from multiple rounds of panning may have high affinity for the target protein. In some cases, one or more of the target polypeptides obtained from multiple rounds of panning may have a dissociation constant (KD) for the target protein of less than about 100 less than about 50 less than about 25 less than about 10 less than about 5 less than about 1 less than about 500 nM, less than about 250 less than about 100 nM, less than about 50 nM, less than about 25 nM, less than about 10 nM, less than about 5 nM, less than about 1 nM, less than about 0.5 nM, less than about 0.1 nM, or less.

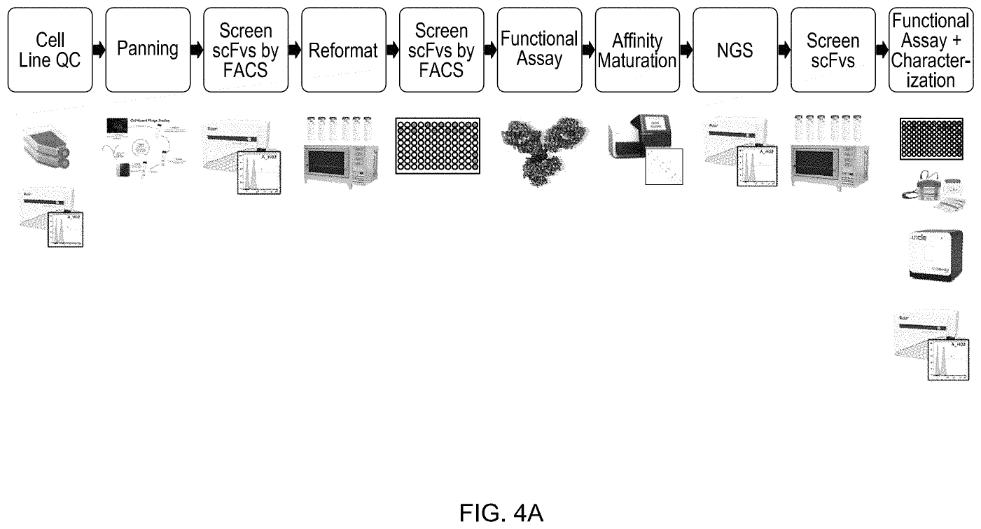

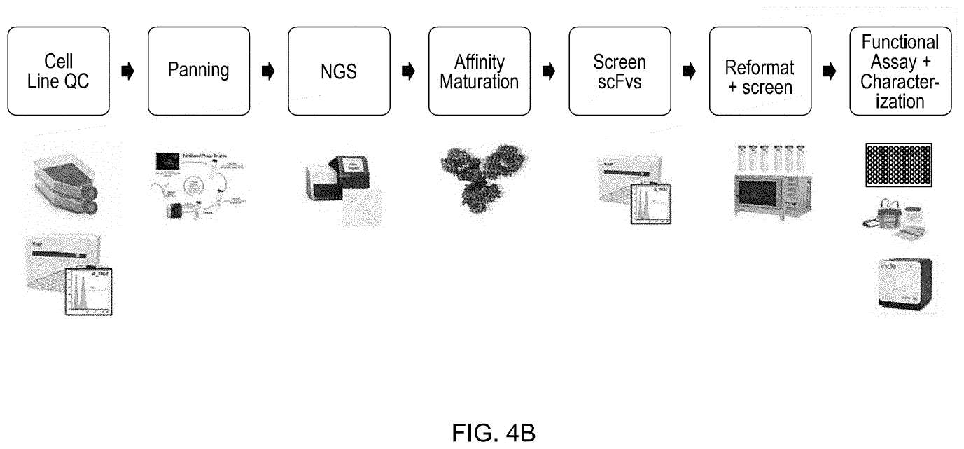

In various aspects, the methods involve additional screening and selecting steps to further improve the affinity of target polypeptides selected from multiple rounds of panning for the target protein. Any additional methods of screening and selecting may be used. FIG. 4A and FIG. 4B, as described in Example 4, provide non-limiting examples of workflows that may be used to further improve the affinity of target polypeptides for the target protein.

In various aspects, the target polypeptides obtained from multiple rounds of panning, as described above, may be screened for binding to the target protein. Any method of screening for binding of a polypeptide to a target protein may be used. In some cases, screening may identify target polypeptides that bind with high affinity for the target protein. In some cases, screening may involve performing flow cytometry to screen for polypeptides that selectively bind to the target protein. In some cases, screening may involve performing next generation sequencing on the polypeptides (e.g., by sequencing polynucleotide tags attached thereto) obtained from the panning rounds to identify target polypeptides that selectively bind to the target protein. In some cases, screening may involve performing one or more functional assays on the target polypeptides. In some cases, the functional assay may be an in vitro assay, an in vivo assay, or an ex vivo assay. In some cases, the functional assay may involve determining whether the target polypeptide has functional activity. For example, the target polypeptide can be tested for its ability to block or inhibit the target protein. In another example, the target polypeptide can be tested for its ability to activate the target protein. In some cases, the target polypeptide may activate or inhibit the target protein in the presence of a ligand. In other cases, the target polypeptide may displace the ligand or compete with the ligand for binding to the target protein.

In various aspects, one or more affinity maturation steps may be performed on target polypeptides obtained from one or more rounds of panning. Affinity maturation may be used to increase the affinity of the target polypeptides for the target protein and/or to find cross-reactive binders. In some cases, affinity maturation may be used to, e.g., thermostabilize the target polypeptide, remove biochemical liabilities from the target polypeptide, pH sensitize the target polypeptide, minimize immunogenicity, prevent polydispersity, prevent aggregation, or any combination thereof. Any suitable method of affinity maturation may be used.

### Target Proteins and Cell Lines Engineered to Express the Same

In some cases, the target protein can be a membrane-bound protein. The membrane-bound protein can be a peripheral membrane protein, wherein the protein is temporarily attached to a biological membrane. Attachment to a biological membrane can comprise attachment to the outer surface of the membrane or can comprise an attachment that includes a domain of the protein spanning the length of the biological membrane. The biological membrane can be a cell membrane. The cell membrane can be a plasma membrane. The cell membrane can be a eukaryotic cell membrane. The cell membrane can be a prokaryotic cell membrane. The membrane-bound protein can be an integral membrane protein, wherein the membrane protein is permanently attached to a biological membrane. The integral membrane protein can be a transmembrane protein.

A transmembrane protein can be a protein comprising at least one domain that spans the length of the cell membrane, also referred to as a transmembrane domain. In some instances, the transmembrane protein comprises at least 2, 3, 4, 5, 6, 7, 8, 9, 10, 11, 12, 13, 14, 15, 16, 17, 18, 19, 20, or more than 20 transmembrane domains. In some instances, the transmembrane protein comprises seven transmembrane domains. The transmembrane protein can be an alpha helical protein or a beta-barrel protein. The transmembrane can be a type I, type II, type III, or a type IV transmembrane protein.

The transmembrane protein can be a cell-surface receptor. The cell-surface receptor can be a membrane transport protein, an enzyme coupled receptor, or a G protein coupled receptor (GPCR). The membrane transport protein can be an ATP powered pump, an ion channel, or a transporter. A transporter can be a uniporter, symporter, or antiporter. The ion channel receptor can be a ligand-gated ion channel receptor or a voltage-gated ion channel receptor. The enzyme coupled receptor can comprise at least one of a kinase, a cyclase, and a phosphatase. The enzyme coupled receptor can comprise at least one activity selected from the following: tyrosine kinase activity, tyrosine phosphatase activity, serine or threonine kinase activity, and guanylyl cyclase activity.

The transmembrane protein can be a G protein-coupled receptor (GPCR). The GPCR can be a Class A, Class B, Class C, Class D, Class D, Class E, or Class F GPCR. A Class A GPCR can be rhodopsin-like receptor. The rhodopsin-like receptor can comprise any receptor in Subfamily A1, A2, A3, A4, A5, A6, A7, A8, A9, A10, A11, A12, A13, A14, A15, A16, A17, A18, or A19. A Class B GPCR can be a GPCR in the secretin receptor family. The secretin receptor family can comprise any receptor in Subfamily B1, B2, or B3. A Class C GPCR can be a metabotropic glutamate receptor, a calcium sensing receptor, a gamma-amino-butyric acid (GABA) type B receptor, a vomeronasal type-2 receptor, a retinoic acid-inducible orphan GPCR (RAIG), or a taste receptor. A Class D GPCR can be a fungal mating pheromone receptor. A Class E GCPR can be a cyclic AMP receptor. A Class F GCPR can be a frizzled GPCR.

In some cases, the target protein may be a protein that is not normally expressed at or on the surface of a cell. In some cases, the target protein may be engineered to include a cell surface localization signal that directs the target protein to the cell surface. In some cases, the target protein may be any artificially expressed surface membrane polypeptide including, but not limited to, CAR, BiTE, VHH, peptide MHC, TCR complex, or T cell antigen coupler (TAC). In some cases, the target protein may be any surface receptor or surface anchoring receptor. In some cases, the target protein may be a glycoprotein (e.g., comprising a plurality of oligosaccharide chains). In some cases, the target protein may be a lipophilic protein. Other non-limiting examples of target proteins include protein complexes such as any homodimer, heterodimer, trimer, tetramer, hexamer, or pentamer receptors, checkpoint proteins, members of the TNF receptor superfamily, integrins, selectins, TCR complexes, MHC, MHC-peptides, cytokine receptors, growth receptors, enzymes, among others. In some instances, the target protein may be expressed on the surface of the second entity. In a particular example, the second entity may be an engineered cell expressing the target protein. In some cases, the cell may be engineered to express the target protein at a high copy. In another example, the second entity may be a cell which naturally expresses the target protein. In some cases, the second entity may be a cell which does not naturally express the target protein. The cell may be a eukaryotic cell. The eukaryotic cell can be a mammalian cell. In some cases, the cell can include a cell line. Example cell lines include, but are not limited to, CHO cells (e.g., CHO-K1 and derivatives thereof such as suspension CHOZN); Human Embryonic Kidney (HEK) cells and any variant or derivative thereof (e.g., HEK293); Caco2 cells; U2-OS cells; NIH 3T3 cells; NSO cells; SP2 cells; DG44 cells; K-562 cells, U-937 cells; MC5 cells; IMR90 cells; Jurkat cells; HepG2 cells; HeLa cells; HT-1080 cells; HCT-116 cells; Hu-h7 cells; HUVEC cells; Molt 4 cells; and BA/F3 cells. The cell can be a stem cell, an embryonic stem cell (ESC), or an induced pluripotent stem cell (iPSC). In some cases, two or more cell lines may be engineered to express the target protein. In some cases, the two or more cell lines may be from different species (e.g., Jurkat or HEK293 from human, and CHO from Chinese hamster). In some cases, the two or more cell lines may be alternated between panning rounds, as described herein. In some cases, the cell line selected to express the target protein may be derived from a parental cell line that may be used as the first entity and/or the third entity in one or more panning rounds as described herein. In some instances, the cell may be a cell from a tumor. The tumor can be a tumor of the bladder, brain, breast, blood, bone, cervix, colon, esophagus, eye, head and neck, heart, kidney, liver, lung, larynx, lymph nodes, ovary, pancreas, prostate, skin, stomach, testicle, rectum, or uterus. Other types of cells that may be used to express the target protein include, without limitation, insect cells, bacterial cells (e.g., E. coli), and yeast cells (e.g., S. cerevisiae). Additional non-limiting expression systems suitable for use with the methods provided herein include mammalian cell display, ribosomal display, and cis-display.

In some cases, the target protein may be engineered to include one or more additional features. Generally, the one or more features should not interfere with folding of the target protein, such that the target protein is expressed in its native conformation. The one or more additional features may be covalently attached to the target protein. In some cases, the one or more additional features are attached to the target protein at an N-terminus. In some cases, the one or more additional features are attached to the target protein at a C-terminus. In some cases, the one or more additional features are expressed at an extracellular portion of the target protein. In some cases, the one or additional features are expressed at an intracellular portion of the target protein. Non-limiting examples of features that may be engineered to be expressed with the target protein are provided herein. In some cases, the C-terminus of the target protein is truncated to prevent internalization of the target protein.

In various aspects, the target protein may comprise a detectable label. In some cases, the detectable label is covalently attached to the target protein. The detectable label may be attached to the C-terminus of the target protein or the N-terminus of the target protein. The detectable label can be a fluorescence marker. The fluorescence marker can be a fluorescent protein. The fluorescent protein can be a green fluorescent protein (GFP), a blue fluorescent protein (BFP), a cyan fluorescent protein (CFP), a yellow fluorescent protein (YFP), an orange fluorescent protein (OFP), or a red fluorescent protein (RFP; e.g., mCherry). The fluorescent protein can be a green fluorescent protein (GFP), or a derivative thereof. The GFP can be a wild type GFP, an enhanced GFP (EGFP), TagGFP, TagGFP2, TurboGFP, Emerald GFP, Monster Green, Azami Green, ZsGreen, hrGFP, Renilla GFP, or Verdi GFP. Non-limiting examples of fluorescent proteins that may be used herein may be found at the web site: fpbase.org/table/. In various aspects, the detectable label may be used to detect target protein expression at the surface of a cell (e.g., by fluorescence microscopy and/or flow cytometry).

In various aspects, the target protein may comprise one or more affinity tags. In some cases, the one or more affinity tags may include a peptide tag or a protein tag. Non-limiting examples of peptide and protein tags include: Au5, AviTag, C-tag, calmodulin-tag, CBP, polyglutamate tag, E-tag, ECS tag, FLAG-tag, Glu-glu, HA-tag, His-tag, KT3, Myc-tag, NE-tag, Rho1D4-tag, S-tag, SBP-tag, Softag 1, Softag 3, Spot-tag, Strep-tag, TC tag, Ty tag, T7 tag, V5 tag, VSV-tag, Xpress tag, isopeptag, SpyTag, SnoopTag, DogTag, SdyTag, biotin carboxyl carrier protein (BCCP), glutathione-S-transferase tag, HaloTag, SNAP-tag, CLIP-tag, maltose binding protein, Nus-tag, thioredoxin-tag, Fc-tag, carbohydrate recognition domain, RFP, and streptavidin. In some cases, the one or more tags may be attached to the N-terminus or the C-terminus of the target protein. In some cases, the one or more affinity tags may be attached to an extracellular portion of the target protein or may be attached to an intracellular portion of the target protein. In various aspects, the affinity tag may be used to detect target protein expression at the surface of a cell (e.g., by staining with an antibody with specificity for the affinity tag).

In various aspects, the target protein may include one or more sequences that localizes the target protein to a cell surface. In some cases, the target protein may include one or more sequences that prevents a target protein from internalizing or being removed from a cell surface. In some cases, the target protein does not comprise a sequence that causes the target protein to be internalized or removed from a cell surface. In one non-limiting example, a target protein of the disclosure may include a Lucy sequence derived from the gene LRRC32 which may localize the target protein to a cell surface and/or prevent internalization of the target protein. In another non-limiting example, a target protein of the disclosure may include a CAR T leader sequence.

In various aspects, the target protein may include any additional features. In some cases, the target protein may include a barcode (e.g., a DNA barcode).

In various aspects, the target protein (including the one or more additional features) may be expressed by the second entity (e.g., a cell) for use in the panning methods described herein. Any suitable method of generating an engineered cell may be used. In some cases, the cell may be engineered to transiently express the target protein. In some cases, the cell may be used to stably express the target protein. In some cases, the target protein may be encoded by a polynucleotide that is introduced into the cell by any method. In some cases, the polynucleotide is introduced into the cell by use of an expression vector. An expression vector may include one or more additional elements that lead to efficient transcription of the polynucleotide encoding the target protein. Such additional elements may include regulatory elements such as promoters, enhancers, and the like. Any suitable expression vector may be used to generate engineered cell lines, including, but not limited to, plasmids, viral vectors (such as lentiviral vectors, adenoviral vectors, adeno-associated viral (AAV) vectors, retroviral vectors, and the like), phage, cosmids, bacterial artificial chromosomes (BACs), yeast artificial chromosomes (YACs), human artificial chromosomes, and the like.

In various aspects, cells may be sorted based on the level of expression of the target protein, prior to being used in the panning methods provided herein. In some cases, the cells may be sorted based on the level of a detectable label attached to the target protein. In a non-limiting example, the cells may be sorted based on the level of fluorescence detected from a fluorescent protein attached to the target protein (e.g., GFP).

### EXAMPLES

### Example 1. Generation of Engineered G Protein-Coupled Receptor (GPCR) Construct

Two DNA constructs were designed to generate engineered GPCRs for use with the methods disclosed herein.

### Construct 1:

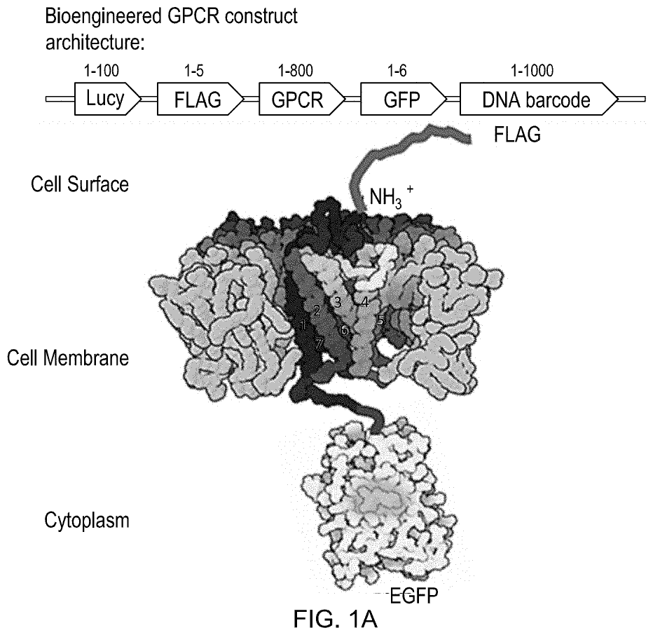

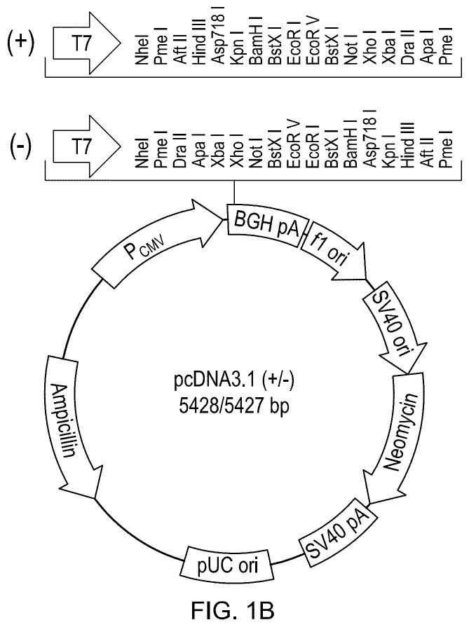

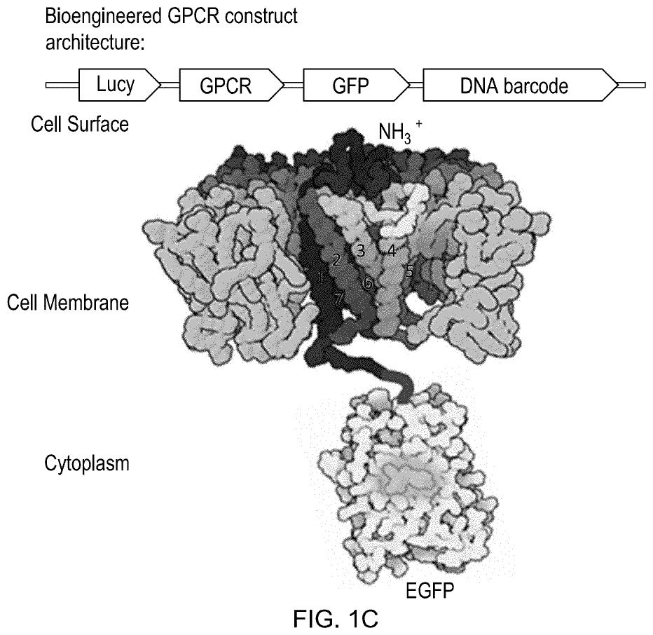

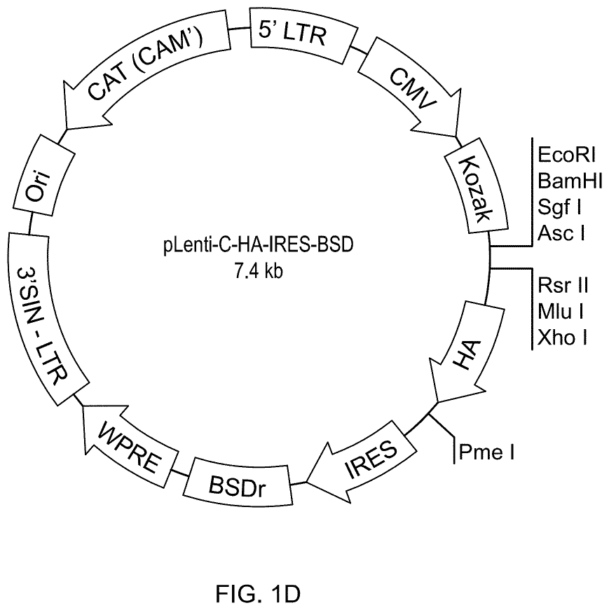

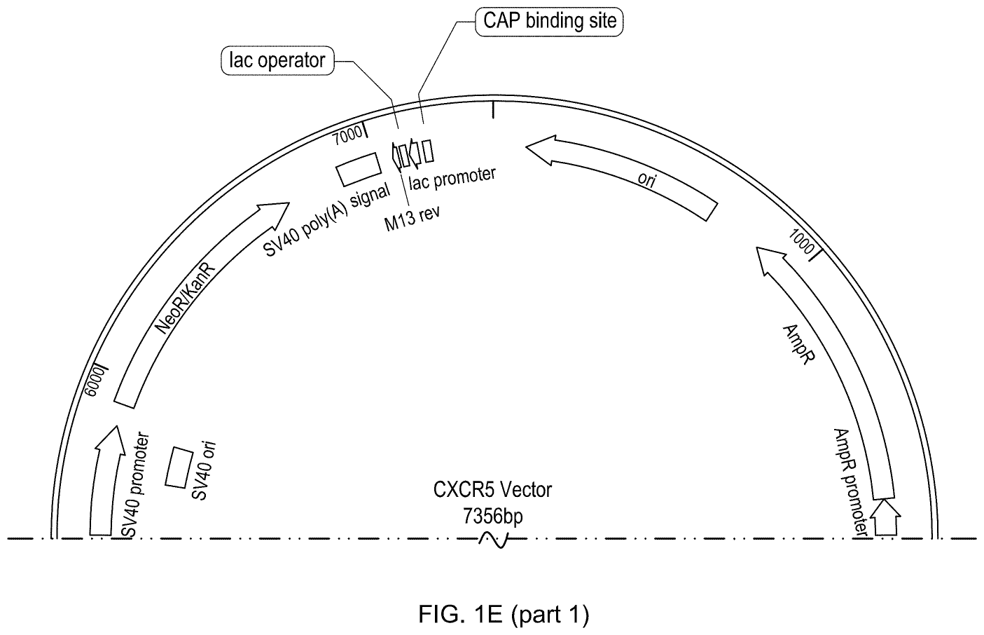

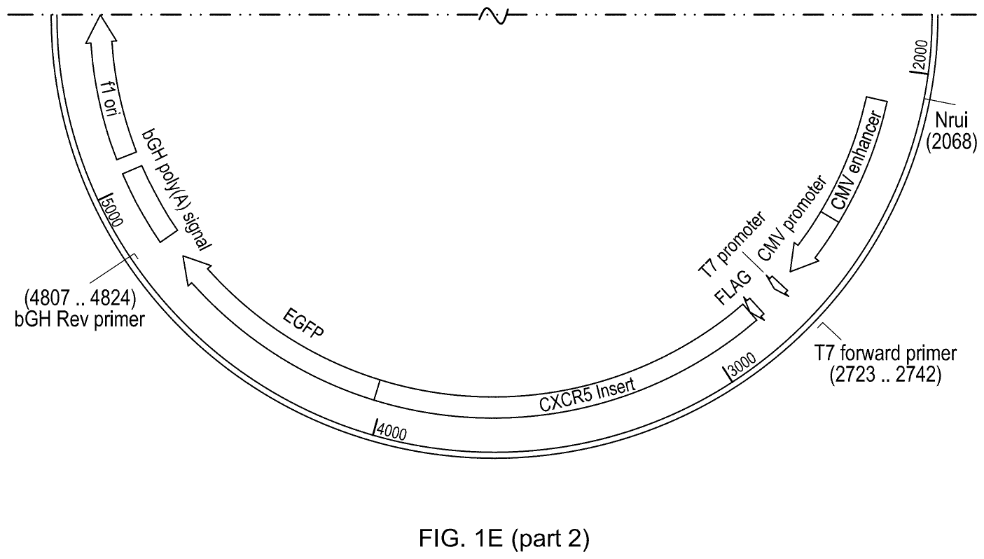

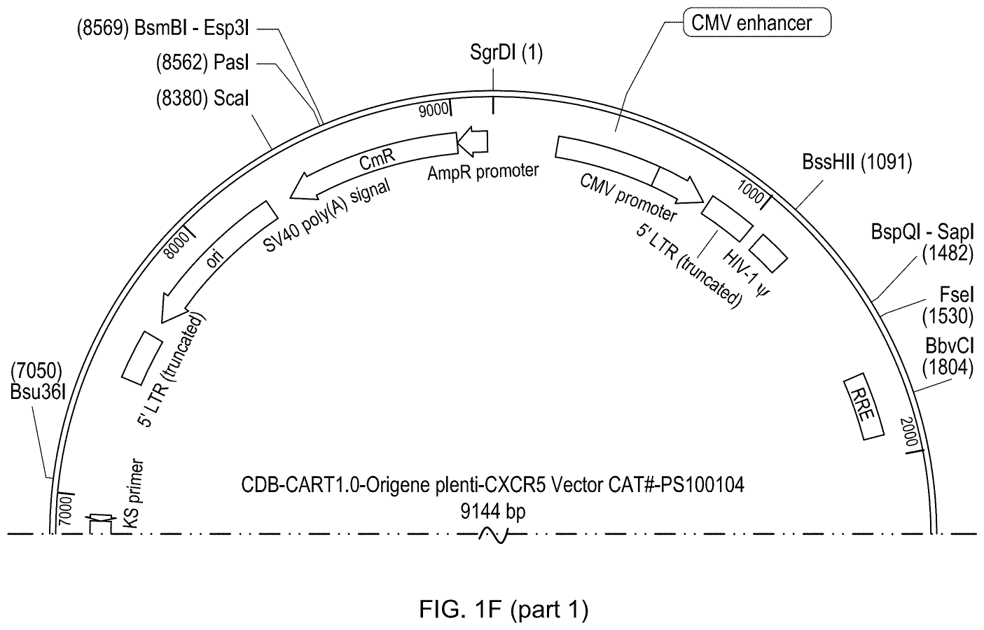

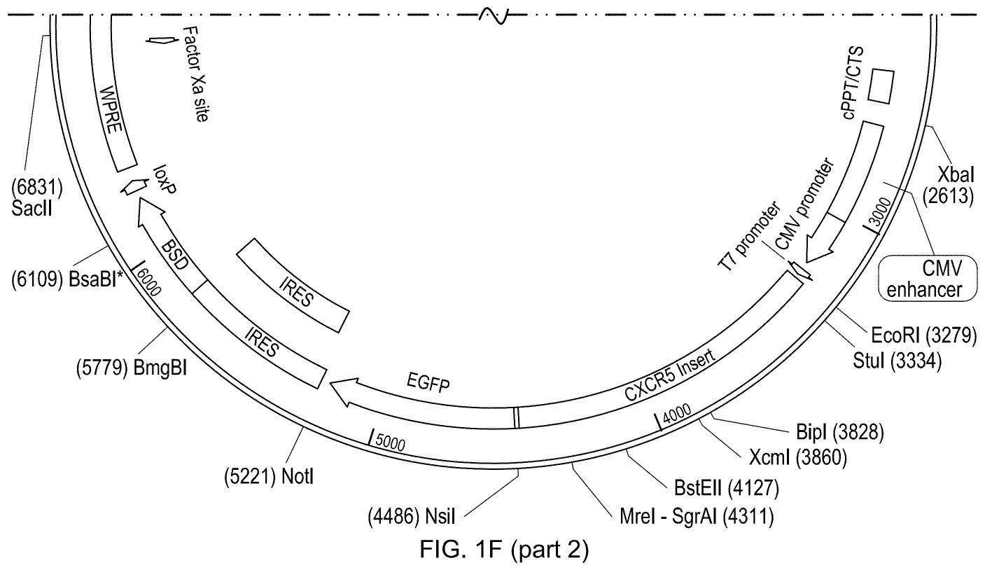

In one example, Construct 1 was designed for stable expression of an engineered GPCR. The DNA construct comprised a nucleic acid sequence encoding an engineered GPCR as depicted in FIG. 1A. The DNA construct included the following components: (1) a nucleic acid sequence encoding a leader sequence “Lucy” from the gene LRRC32. This sequence was responsible for surface localization of the GPCR target; (2) a nucleic acid sequence encoding a FLAG tag which enabled the detection of membrane localization when analyzing cell lines. In some cases, other tags may be used. In some cases, no tag may be used; (3) a nucleic acid sequence encoding one or more glycine-serine (GS) linkers that may encompass different GS sequences and lengths; (4) one or more restriction enzyme sites throughout the insert which were added in frame to clone different GPCR genes; (5) a nucleic acid sequence encoding the target protein. Any protein may be engineered and expressed by this construct. In some cases, the target protein is a GPCR. In this example, the open reading frame of CXCR5 was used; (6) a nucleic acid sequence encoding a fluorescent protein attached to the GPCR construct at the C-terminus. Any fluorescent protein may be used (e.g., green fluorescent protein, blue fluorescent protein, or red fluorescent protein). The fluorescent protein may serve as a marker for detecting expression and translocation to the cytoplasmic membrane. In this example, the fluorescent protein was enhanced green fluorescent protein (EGFP); and (7) a DNA barcode. In some cases, any arbitrary DNA sequence may be used to specifically label a protein. The DNA barcode may be used to identify the cloned DNA post-cell line generation.

The full nucleic acid sequence of Construct 1 encoding an engineered CXCR5 was as follows:

(SEQ ID NO: 1)
5′-ATGAGACCCCAGATCCTGCTGCTCCTGGCCCTGCTGACCCTAGGC
CTGGCGACTACAAGGACGATGACGACAAGGGTTCAGGCAGTGGTTCCGGGT
CAGGGGGAGGTACCATGAACTATCCGCTGACTCTGGAAATGGATCTGGAAA
ATCTCGAAGATCTCTTCTGGGAACTGGACCGGTTGGATAACTACAATGACA
CAAGTCTCGTCGAGAACCACCTGTGTCCAGCTACCGAAGGGCCTTTGATGG
CCTCTTTTAAGGCTGTGTTTGTGCCTGTAGCCTATAGCCTCATTTTCCTCC
TCGGAGTTATTGGAAATGTACTCGTGTTGGTAATCCTTGAGAGACACCGGC
AAACAAGGAGCTCAACTGAAACCTTCCTCTTCCATCTGGCTGTCGCGGATC
TCCTCCTCGTGTTTATCCTTCCATTCGCAGTTGCGGAGGGTTCAGTGGGAT
GGGTGCTCGGAACATTCTTGTGTAAGACTGTGATTGCACTCCATAAGGTCA
ATTTCTACTGCTCCAGTTTGCTGCTCGCCTGCATCGCTGTTGACAGGTATC
TCGCCATCGTACATGCCGTGCACGCATATCGACACAGAAGACTGCTGTCCA
TCCATATTACCTGTGGCACAATTTGGCTGGTGGGATTCCTGCTGGCACTGC
CCGAGATCCTGTTCGCCAAGGTCAGTCAGGGACATCACAATAACTCCCTCC
CACGCTGCACTTTCAGTCAAGAGAATCAGGCAGAAACCCACGCGTGGTTTA
CGTCTCGATTCCTTTACCATGTAGCAGGGTTTCTCTTGCCCATGCTGGTTA
TGGGATGGTGCTACGTTGGAGTAGTTCACAGGCTGCGGCAAGCTCAACGAA
GACCGCAGCGGCAAAAAGCCGTCAGAGTGGCTATCCTTGTCACTTCCATCT
TCTTTCTGTGCTGGAGTCCTTATCACATTGTGATATTCCTGGACACACTGG
CCAGGCTGAAAGCCGTCGATAACACATGCAAGCTCAATGGATCCCTGCCTG
TTGCTATCACAATGTGCGAATTTCTGGGTCTTGCCCATTGCTGTCTGAATC
CTATGTTGTATACCTTCGCTGGCGTGAAATTCCGGAGTGACCTCTCAAGAT
TGCTTACTAAGTTGGGCTGTACAGGCCCCGCTTCTCTGTGTCAGTTGTTTC
CTTCATGGCGACGGTCCAGCCTTAGTGAATCTGAGAACGCTACTAGCCTCA
CCACTTTCGGAAGCGGTTCTGGCAGTGGGAGTATGCATATGGTGAGCAAGG
GCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCCG
TGCCCTGGCCCACCCTCGTGACCACCCTGACCTACGGCGTGCAGTGCTTCA
GCCGCTACCCCGACCACATGAAGCAGCACGACTTCTTCAAGTCCGCCATGC
CCGAAGGCTACGTCCAGGAGCGCACCATCTTCTTCAAGGACGACGGCAACT
ACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACA
AGCTGGAGTACAACTACAACAGCCACAACGTCTATATCATGGCCGACAAGC
AGAAGAACGGCATCAAGGTGAACTTCAAGATCCGCCACAACATCGAGGACG
GCAGCGTGCAGCTCGCCGACCACTACCAGCAGAACACCCCCATCGGCGACG
GCCCCGTGCTGCTGCCCGACAACCACTACCTGAGCACCCAGTCCGCCCTGA
GCAAAGACCCCAACGAGAAGCGCGATCACATGGTCCTGCTGGAGTTCGTGA
CCGCCGCCGGGATCACTCTCGGCATGGACGAGCTGTACAAGAAGCTTTGTG
CACGACGATGAT-3′

Construct 1 was cloned into the pcDNA3.1+ vector (FIG. 1B) for stable expression in a host cell line (GPCR-pcDNA3.1). FIG. 1E depicts Construct 1 cloned into the pcDNA3.1+ vector. As shown in FIG. 1A, Construct 1 encoded an engineered CXCR5 protein having the following structure: from N-terminus to C-terminus, a Lucy signal for directing the protein to a cell surface, an extracellular FLAG tag attached to the N-terminus of CXCR5, the CXCR5 protein, an intracellular GFP tag attached to the C-terminus of CXCR5, and a DNA barcode attached to the C-terminus of the GFP.

### Construct 2:

Construct 2 was similar to Construct 1, except that Construct 2 lacked the extracellular FLAG tag. FIG. 1C depicts a schematic of an engineered CXCR5 protein encoded by Construct 2. Construct 2 was cloned into a pLenti vector (pLenti-C-HA-IRES-BSD from ORIGENE®) (FIG. 1D) for lentiviral transduction in a host cell line. FIG. 1F depicts Construct 2 cloned into the pLenti vector. As shown in FIG. 1C, Construct 2 encoded an engineered CXCR5 protein having the following structure: from N-terminus to C-terminus, a Lucy signal for directing the protein to a cell surface, the CXCR5 protein, an intracellular GFP tag attached to the C-terminus of CXCR5, and a DNA barcode attached to the C-terminus of the GFP.

### Example 2. Sorting Stable Cell Lines and Cells Lines Transduced with Lentivirus

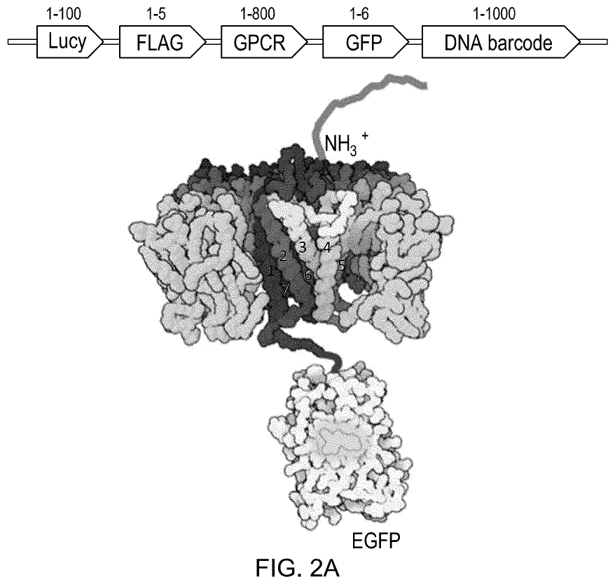

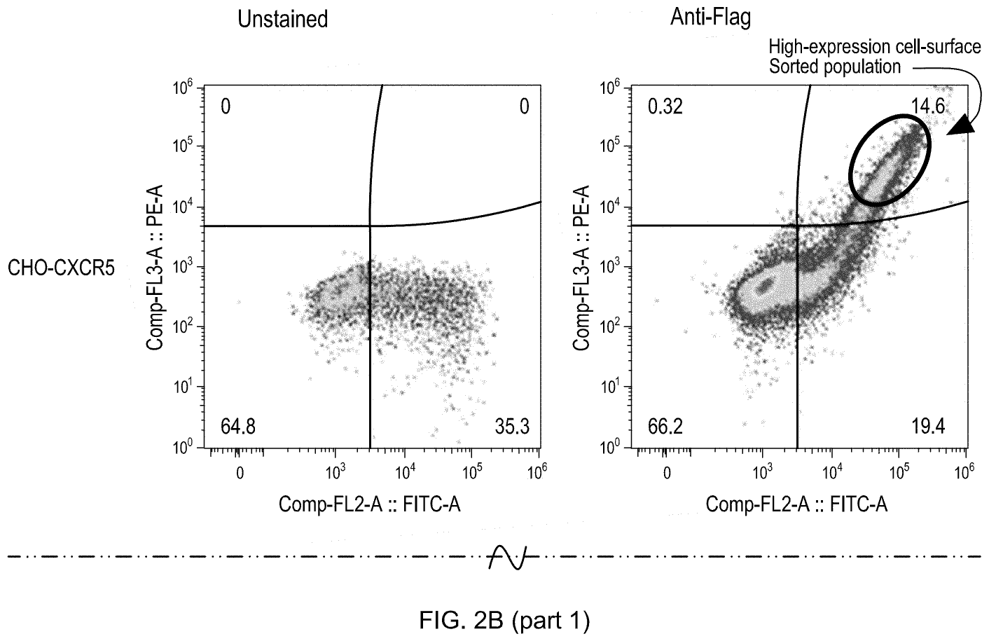

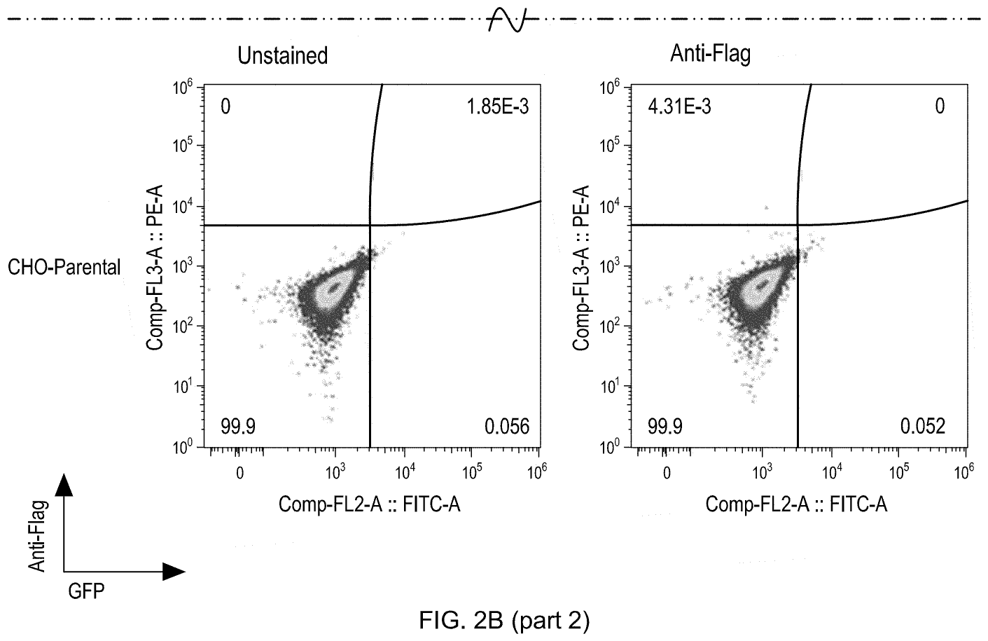

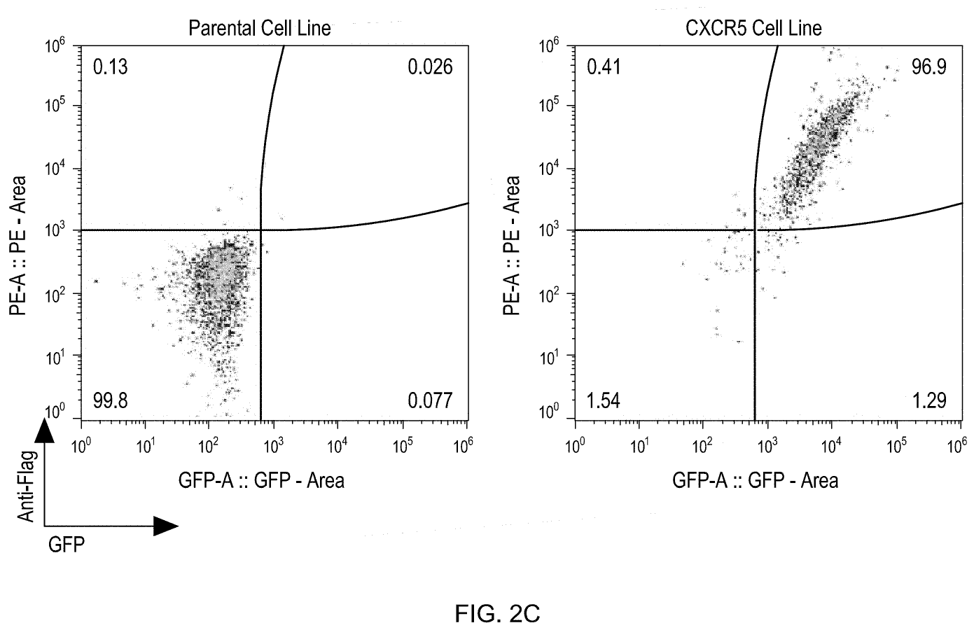

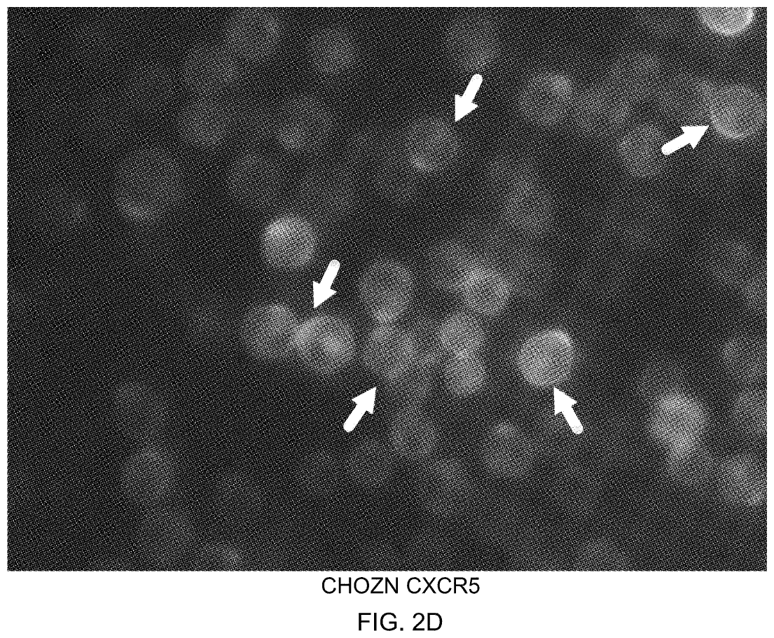

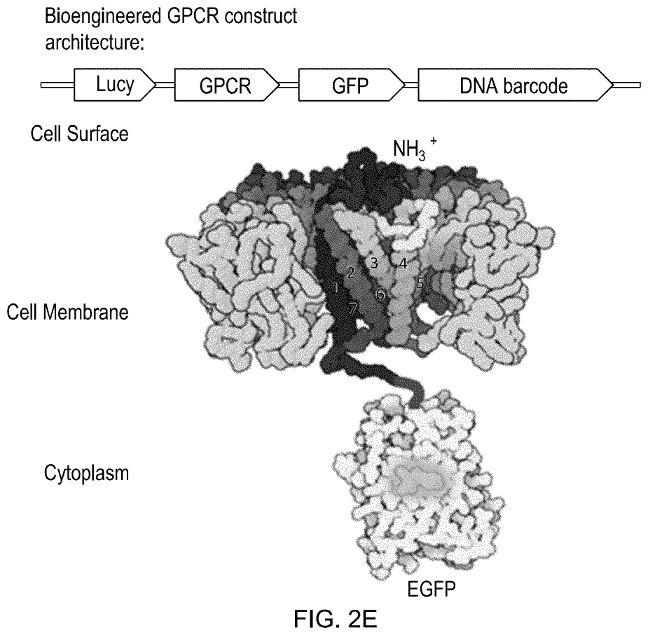

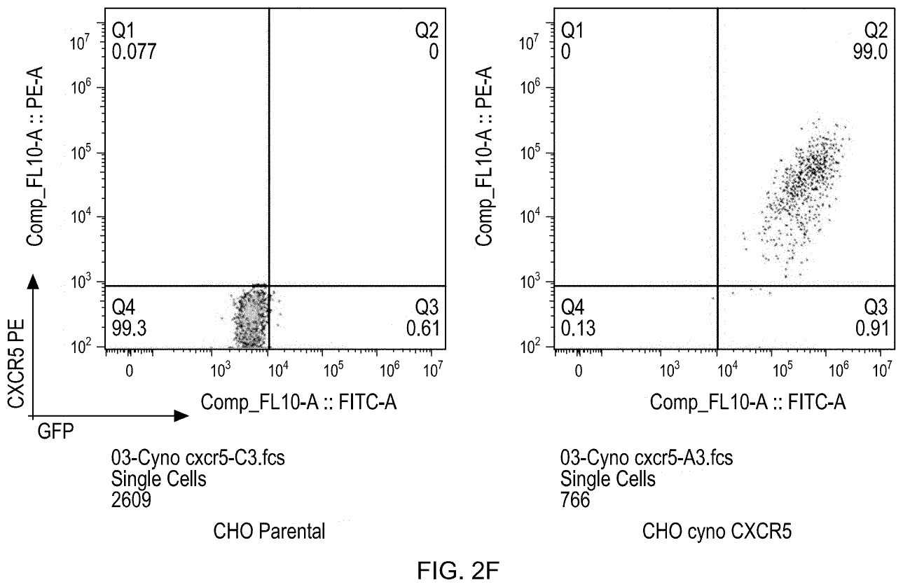

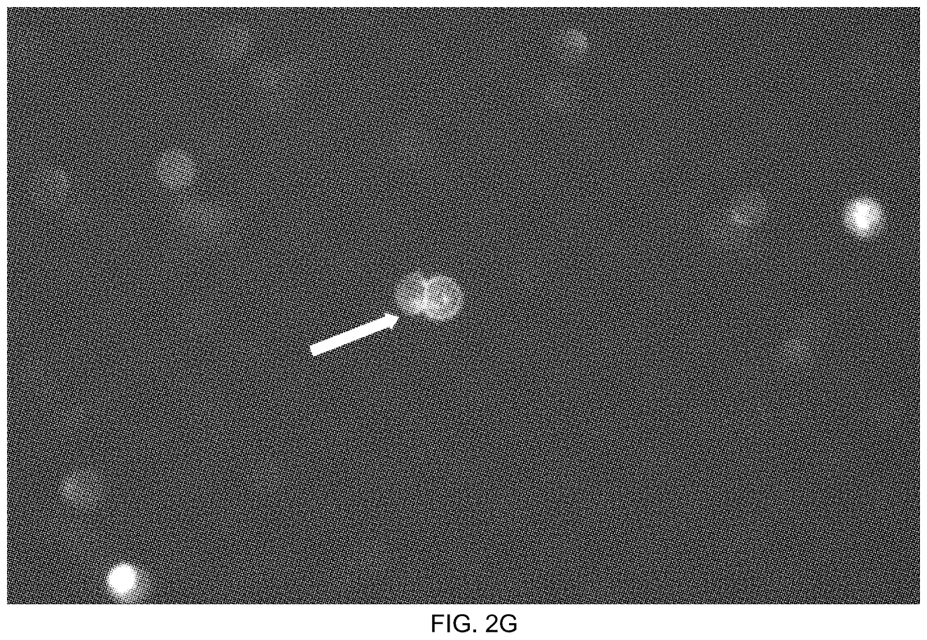

Mammalian cells (e.g., HEK293, CHOZN, CHOK1, and Jurkat) were transfected with GPCR-pcDNA3.1 vector for stable expression using INGENIO® electroporation kit from Mirus Biosciences. FACS plots showed high co-expression of CXCR5 (stained with PROZYME® PHYCOLINK® anti-FLAG-R-Phycoerythrin) and GFP on the cell surface, as shown in FIGS. 2A-2C. FIG. 2D depicts fluorescent microscopy of CHOZN cells showing EGFP fluorescence on the surface of the cell membrane which indicated translocation of the construct to the cell surface (shown by the white arrows).

Lentiviral vectors were generated by transfecting HEK 293TN cells with GPCR-pLenti along with lentivirus packaging plasmids (pPACKH1-XL HIV; SBI System Biosciences). Viral vectors were harvested and used to transduce various cell lines, such as CHOZN, HEK293, and Jurkat cell lines. The lenti-vector construct was stably integrated into the genome of the target cells for long-term expression. FACS plots showed high co-expression of CXCR5 (stained with anti-CXCR5 Monoclonal Antibody Clone: MU5UBEE from eBioscience conjugated with PE) and GFP on the cell surface, as shown in FIGS. 2E and 2F. Fluorescent microscopy of lenti transduced CHOZN cells showed EGFP fluorescence on the surface of the cell membrane indicating translocation of the construct to the cell surface (indicated by white arrow) (FIG. 2G).

### Example 3. Cell Panning to Identify Antibody Fragments that Bind to CXCR5

FIG. 3A depicts an example of a cell panning methodology as described herein. The cell lines expressing the desired GPCR target were sorted to select those cells expressing a high copy number per cell (above 200K copies per cell). For each round of panning, two different host cell lines from different species were alternated (e.g., CHO (hamster) and Jurkat (human)).

An antibody phage library (SuperHuman 2.0 phage library) was depleted by incubating the antibody library with a parental cell line (not expressing CXCR5). In this step, antibodies that were off-target, sticky, and not specific to CXCR5 were removed from the pool. After three rounds of first depletion, the stable cell line was incubated with the depleted antibody pool for the selection round, to select for antibody fragments that bound to CXCR5. In parallel, the parental cell line was incubated with the depleted antibody pool to identify non-specific binders and to eliminate them from the final pool of antibodies (e.g., a second depletion step). After the selection step, the cells were washed with wash buffer (1× PBS+0.5% BSA). After washing, the cells were optionally sorted to select the top 1 million cells and collect the EGFP expressing cells. Next, the phage expressing the pool of positive binders was eluted by resuspending the cells in triethylamine in water and incubated for 10 minutes at room temperature with rotation. After incubation, 1 mL of 1M Tris-HCl pH 7.4 was added to neutralize and the eluted phage were used to infect 10 mL of electrocompetent E. coli cells per condition. After infection, the E. coli with the scFv phage positive pool were plated, the phage clones were amplified, and another round of panning was conducted.

The panning rounds were repeated 4-5 times. For each round of panning, a different host cell line was used from the previous round (e.g., CHO in round 1, Jurkat in round 2, CHO in round 3, and Jurkat in round 4). For each round of first depletion, the total number of cells and the incubation time were the same for the first depletion step. For each round of selection, increasingly more stringent conditions were used in subsequent rounds. For example, in one round of selection, two times more washes and 1/10 the number of cells were used as compared to a preceding round. In another example, in one round of selection, four times more washes and 1/100 the number of cells were used as compared to a preceding round.

After 4-5 rounds of panning, positive clones were selected and clones were screened by either next generation sequencing (NGS) or flow cytometry. Positive clones were reformatted into a full IgG backbone and subjected to appropriate functional assays.

### Example 4. Cell Panning Workflows

Two different strategies were used to identify functional GPCR binders. In a first non-limiting strategy, as depicted in FIG. 4A, cell lines were subjected to quality control, then the panning process was initiated as described in Example 3. Post-round 4 of panning, scFv clones were picked and screened by flow cytometry using one of the cell lines expressing the target protein versus the parental cell line. This FACS screen was used to determine scFvs that bound specifically to the target antigen. Binders were then reformatted into an IgG backbone (e.g., human or mouse). After reformatting, characterizations of melting temperature, temperature of aggregation, and poly dispersity index were measured using Uncle (UNCHAINED LABS®). The dissociation constant (KD) was tested on the cells, and the selected antibodies were tested in functional assays as described below. In parallel, next generation sequencing was carried out directly on the final phage output of round 3 and round 4 of panning. This was used to detect new clones that were binders but did not show up in the primary screen. All clones were reformatted and tested. Once functional antibodies were confirmed, affinity maturation was conducted, if needed, on the desired clones as described in Example 5. Affinity maturation was used to increase the affinity of binders, and to find cross-reactive binders. In the affinity maturation step, different arms were panned against where one arm could be, without limitation, human-cynomolgus-human-cynomolgus or human-mouse-human-mouse. Clones were selected from NGS and the process of screening, reformatting, and testing for functionality and characterization was repeated.

In a second non-limiting strategy, as depicted in FIG. 4B, cell lines were subjected to quality control and the panning process was initiated as described in Example 3. Post-round 4 of panning, NGS was carried out directly on the final phage output of round 3 and round 4 screens. NGS was used to detect all clones that were enriched for the target protein panning arm and parental panning arm. By eliminating the clones enriched in the parental cell line arm, target specific clones were chosen. These clones went directly into a round of affinity maturation, as described in Example 5. Affinity maturation was used to increase the affinity of binders, and to find cross-reactive binders. In the affinity maturation step, different arms were panned against where one arm could be, without limitation, human-cynomolgus-human-cynomolgus or human-mouse-human-mouse. Clones were chosen from another round of NGS and the process of screening, reformatting, and testing for functionality and characterization were repeated.

To measure equilibrium dissociation constant, cells expressing the target protein and the parental cells were incubated in 96-well plates in 100 μL of FACS buffer. 100 μL of the diluted antibody was added to each well in 1:3 dilution format (100 μg/mL, 33 μg/mL, 11 μg/mL, etc.), and cells were incubated on ice for up to 4 hours to establish steady state condition. After incubation, the cells were washed once with FACS buffer and an APC-labeled secondary anti-IgG was added at a 1:1000 dilution. The secondary stain was incubated for 30 minutes on ice and washed twice with FACS buffer. After incubation, the cells were resuspended in 200 μL of FACS buffer and analyzed on a flow cytometer. The data generated from this experiment was analyzed via GraphPrism.

### Example 5. Affinity Maturation of Selected Clones

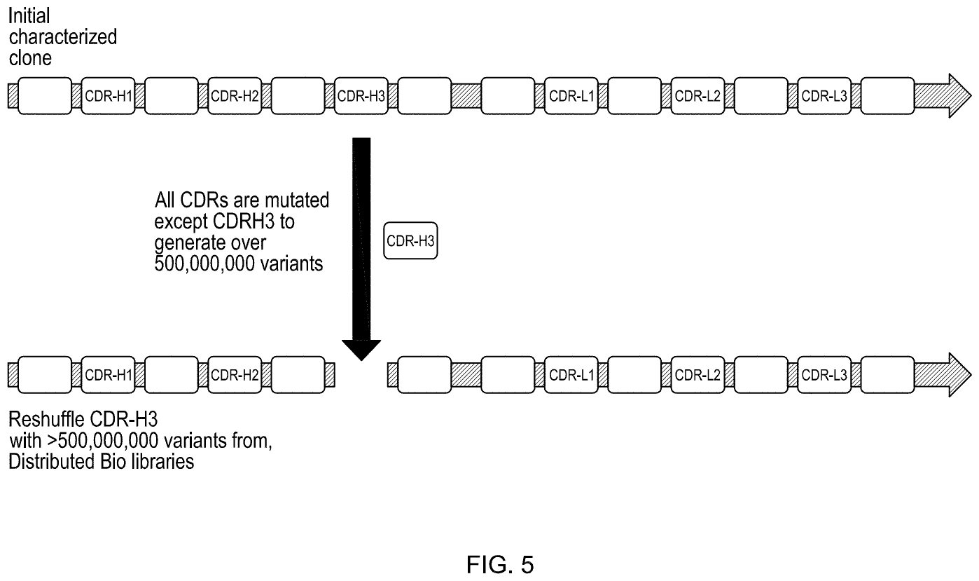

The purpose of affinity maturation was to improve thermostability, deimmunize, species-cross react, remove any biochemical liabilities, humanize, and pH sensitize initial clones that were characterized by the initial panning method. FIG. 5 depicts a non-limiting example of an affinity maturation step. In this example, the CDR-H3 of the initial clone was reshuffled with other variants in the SuperHuman2.0 library. This enabled 500 million opportunities to improve various properties and characteristics of the initially discovered clone. After the affinity matured library was built, the process of cell-based phage display panning was repeated to find higher affinity and improved clones.

### Example 6. Screening Antibody Clones Selected from Cell Panning

### Next Generation Sequencing

Round 3 and Round 4 panning outputs were deep sequenced by NGS. Glycerol stocks were miniprepped using Qiagen Miniprep kit and eluted in 100 μl of elution buffer provided in the kit. Following miniprep, a PCR reaction was performed to barcode the VH and FW3 fragments separately. For the forward primers, barcodes md01-md04 were used, and for the reverse primers, barcodes md01-md12 were used. Following the barcode PCR reaction, samples were run on a 2% agarose gel, extracted, and cleaned. After barcoding was completed, another PCR reaction was run on the barcoded samples to add the PE adapters. Two specific primers, optPE1 and optPE2, were used. All primers were used at a 10 μM concentration. After the PE adapters were added, samples were run on a 2% agarose gel, extracted, and cleaned. Concentrations were measured and samples were pooled based on the concentrations and the number of reads desired from the run. Once the sample pool was ready it was loaded onto a MISEQ® (Illumina).

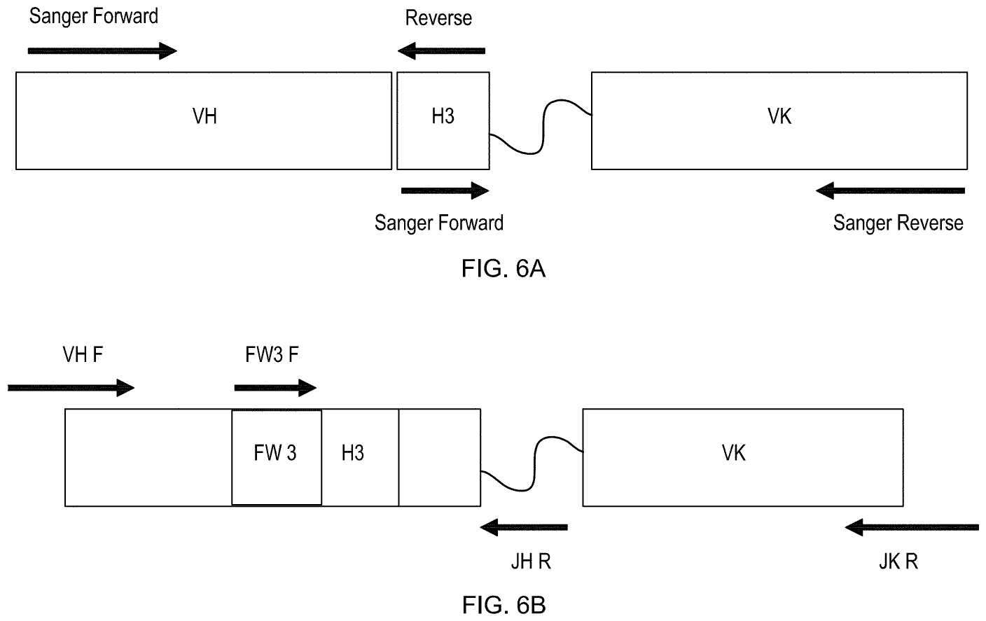

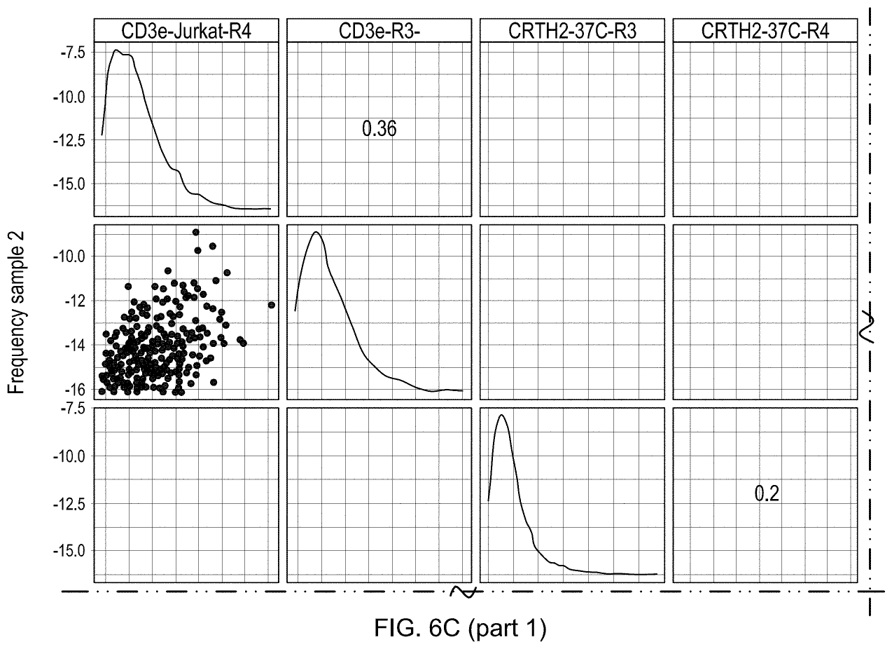

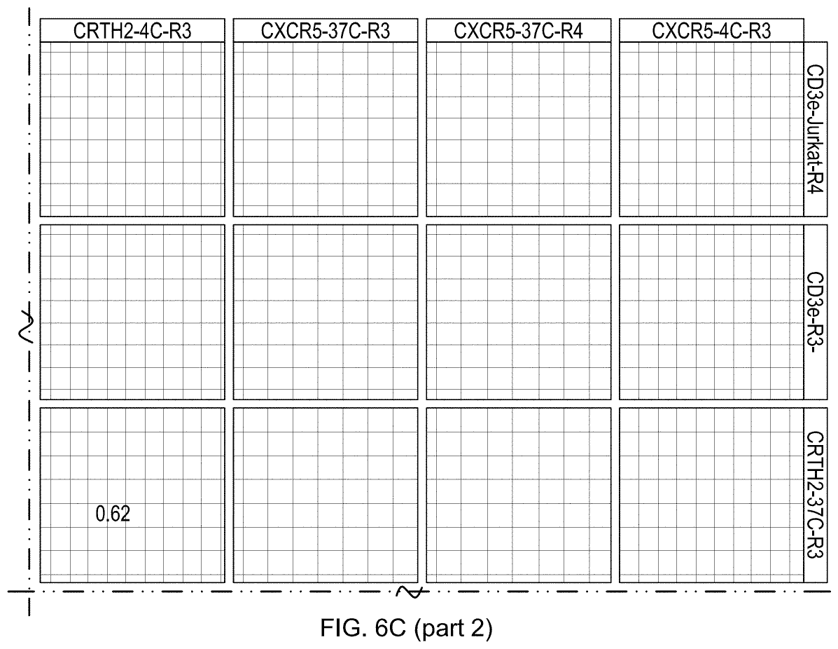

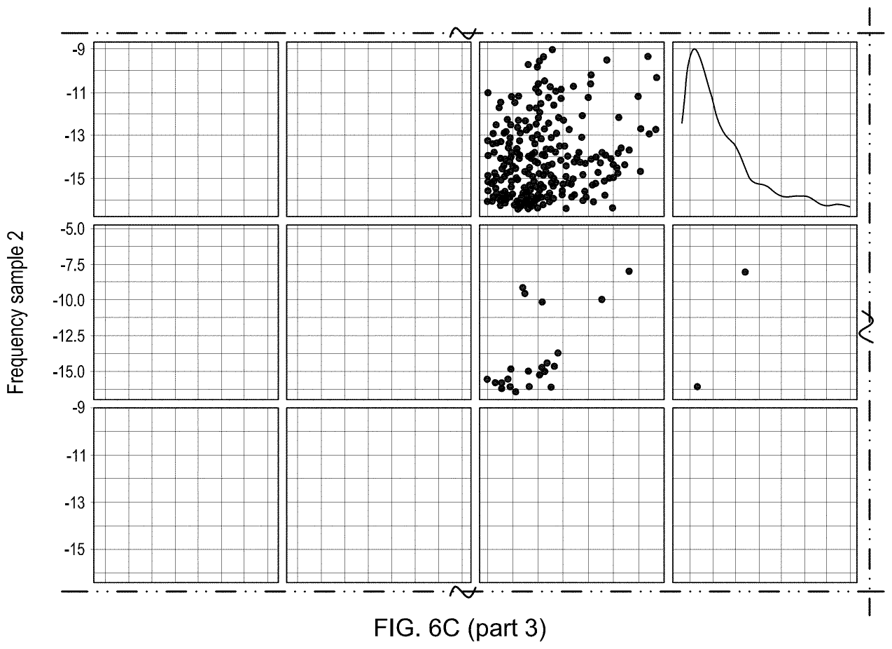

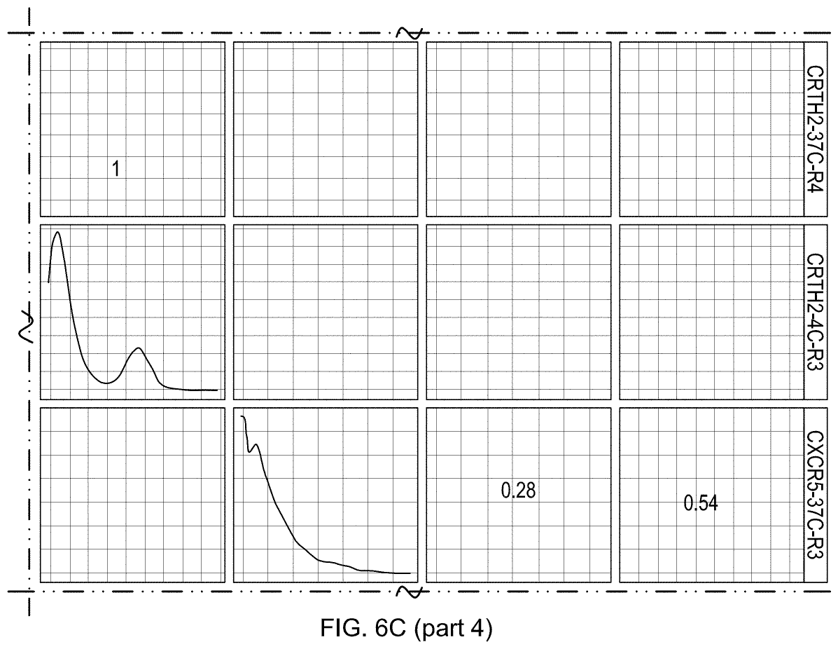

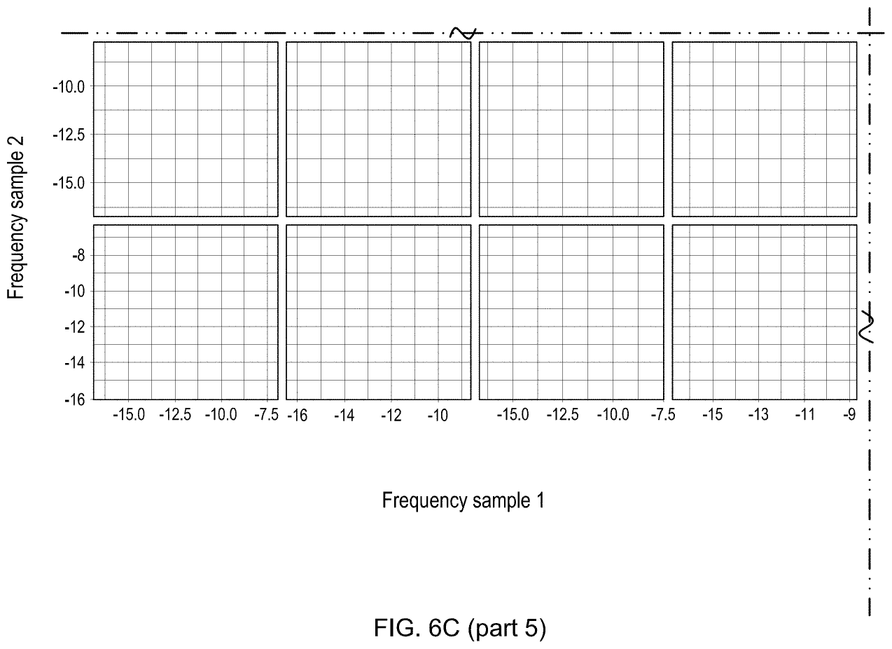

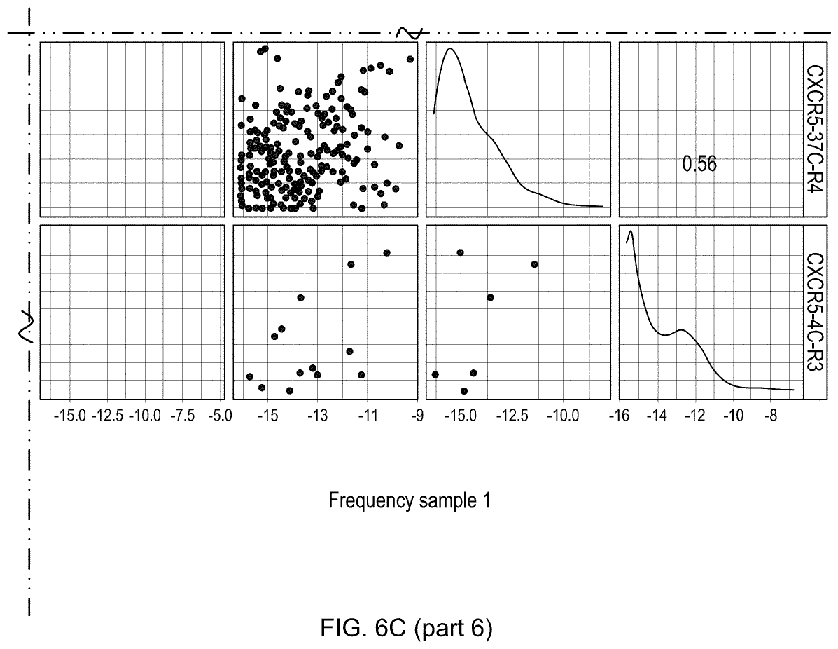

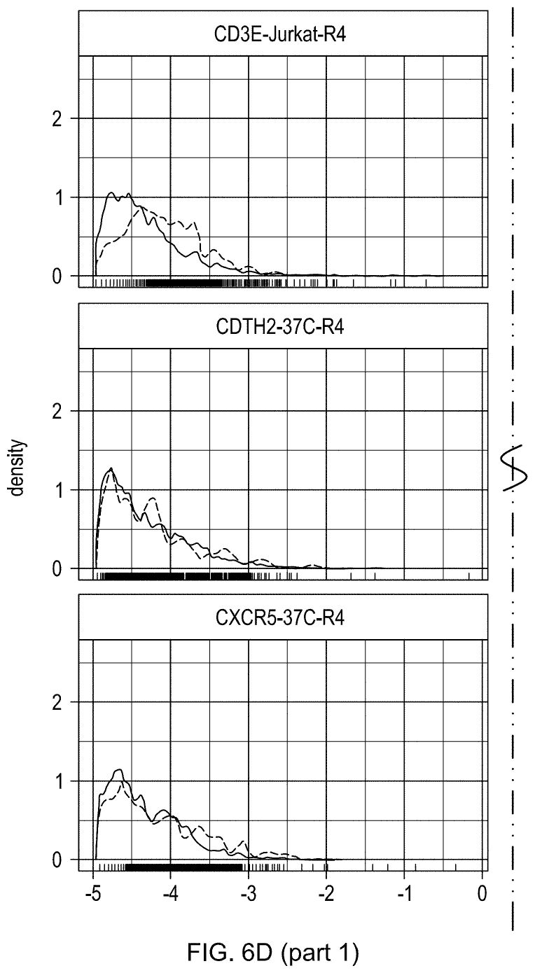

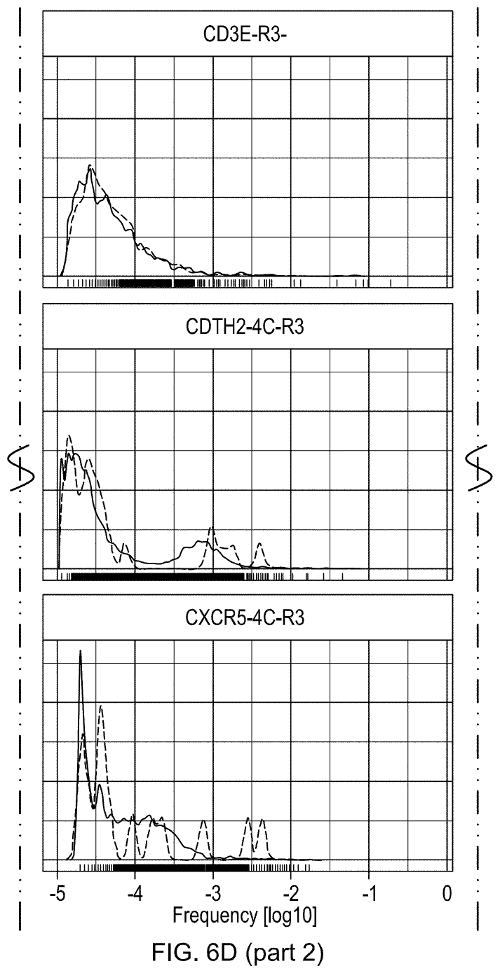

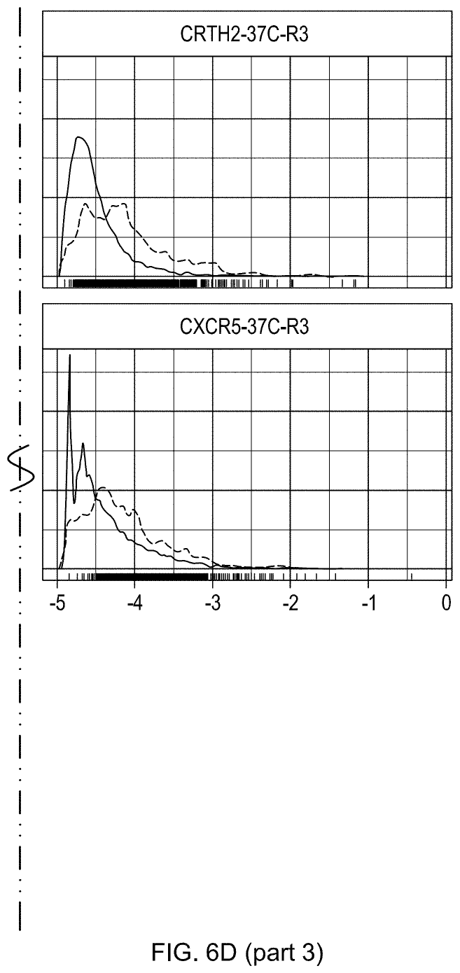

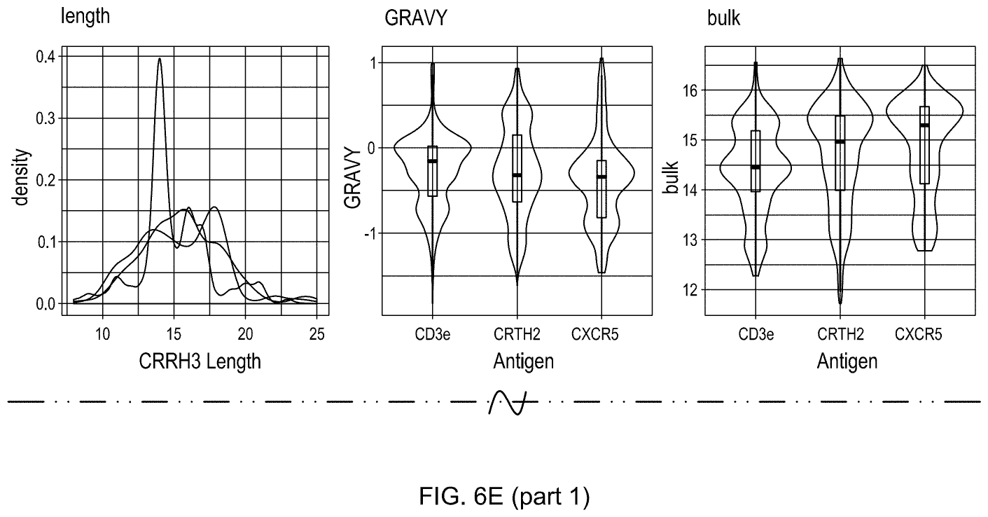

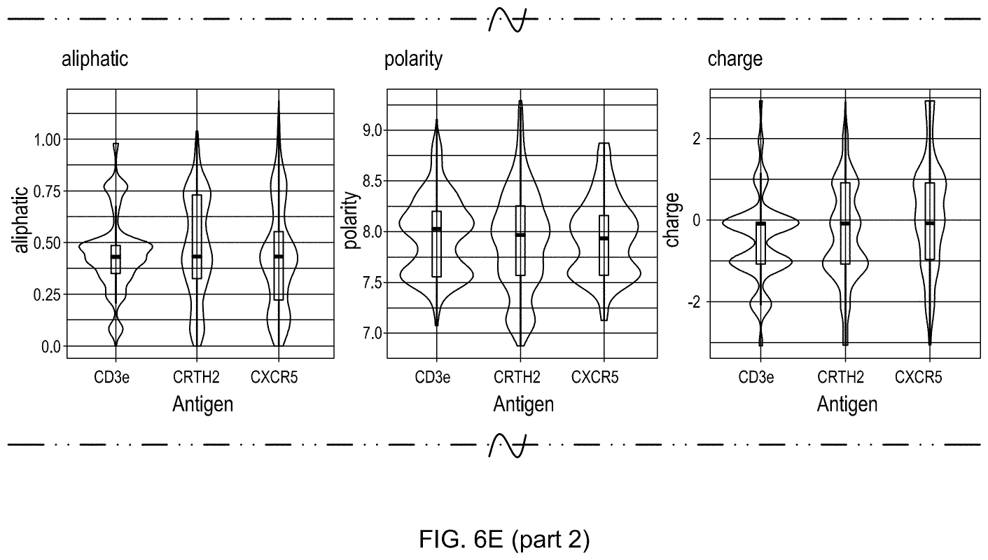

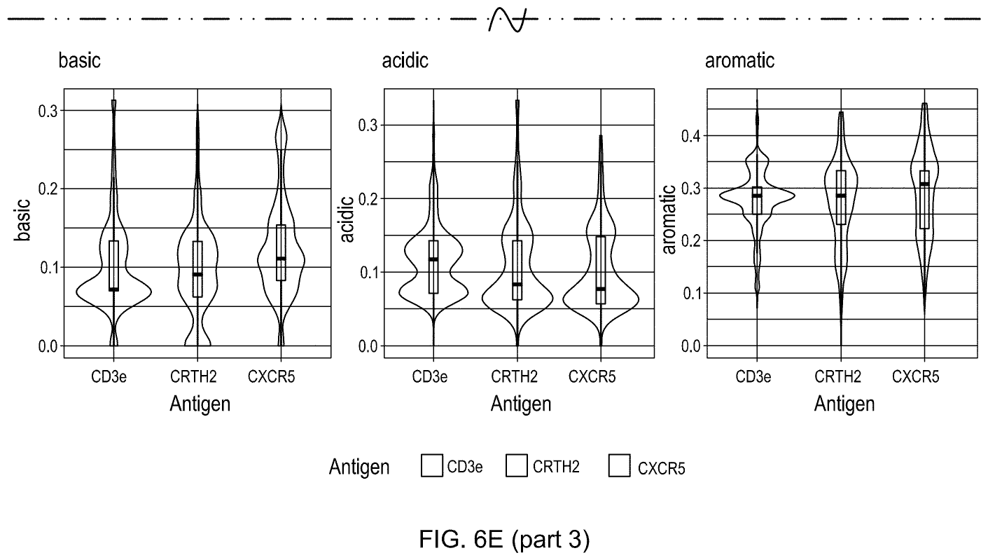

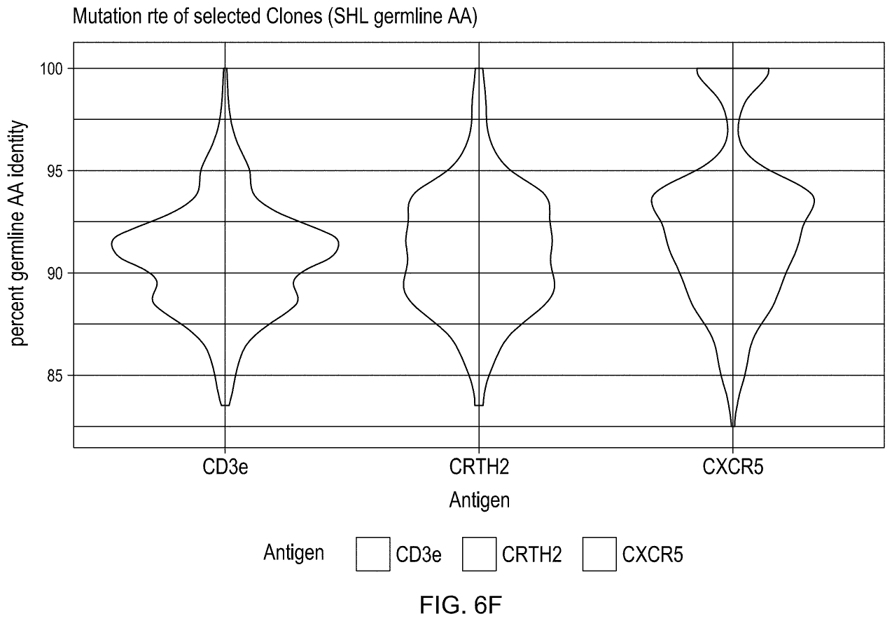

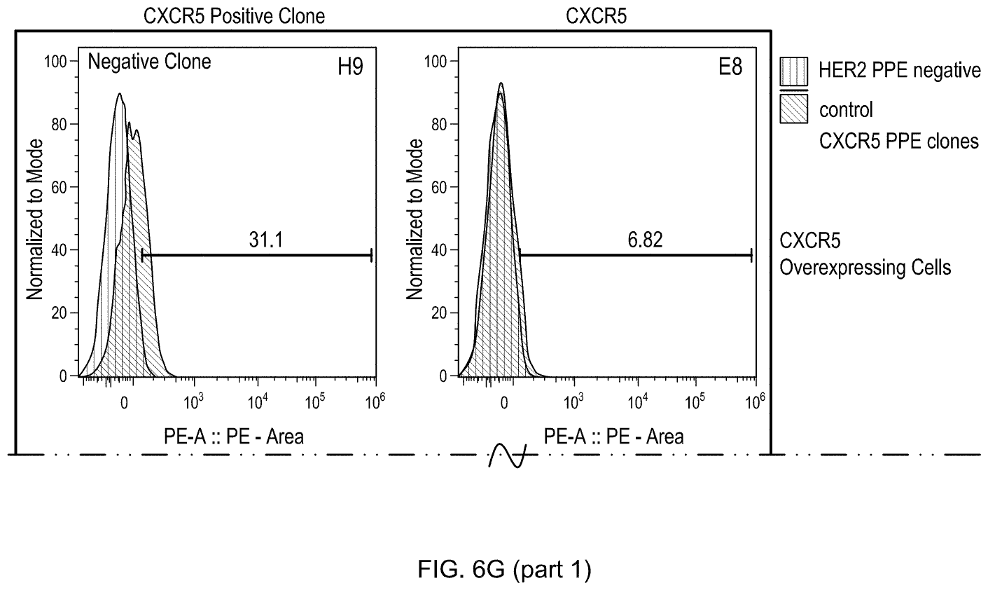

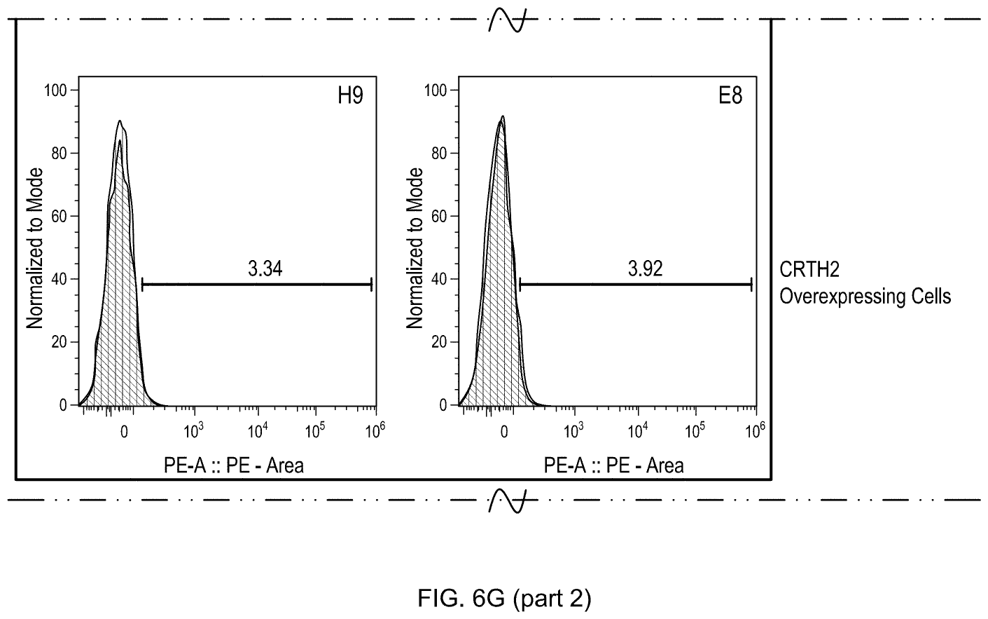

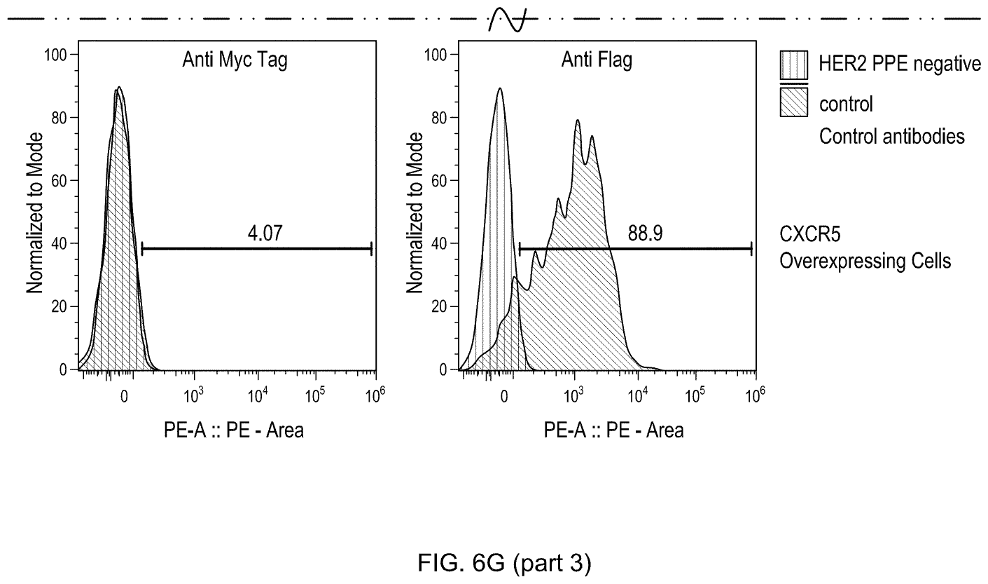

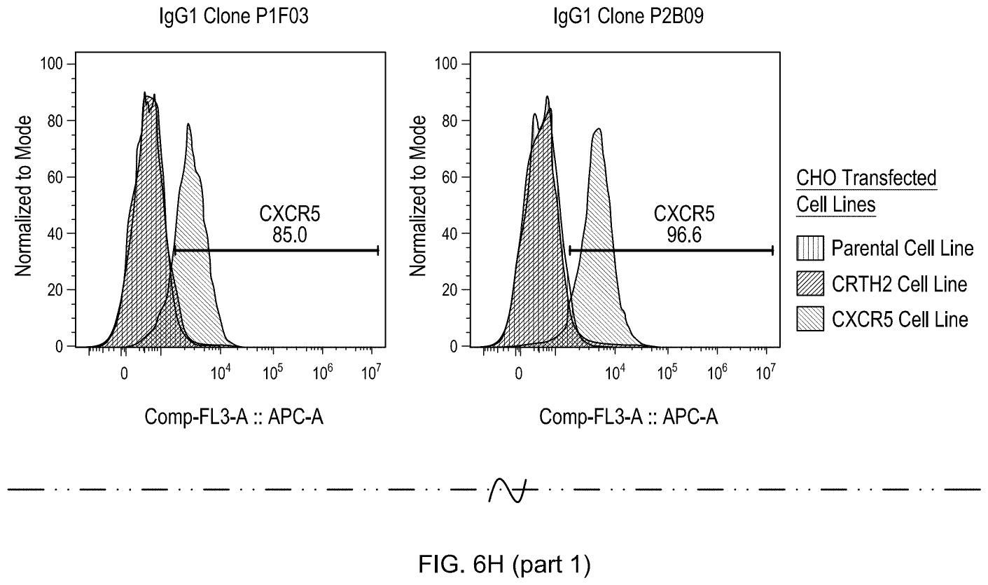

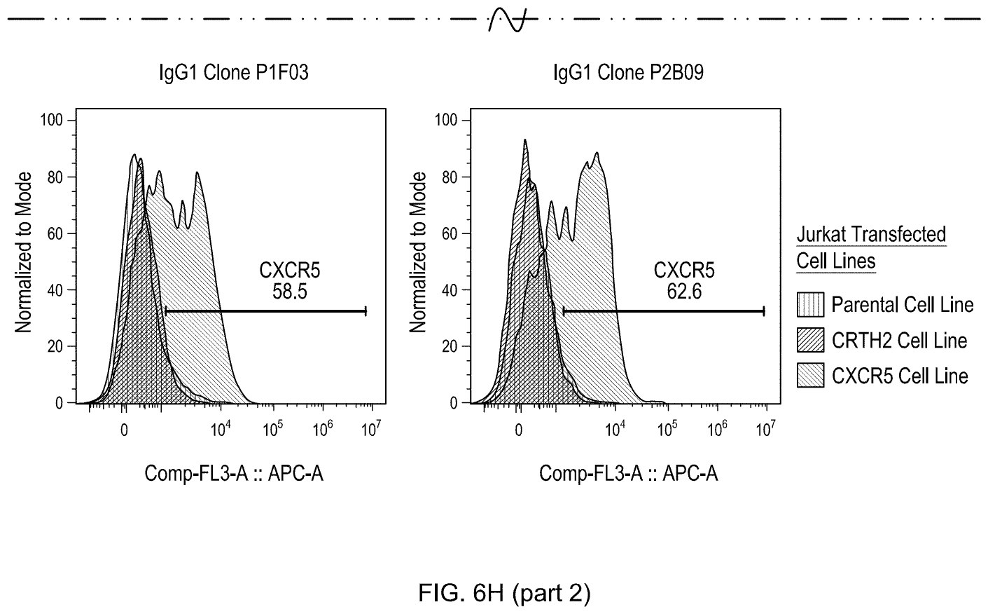

FIG. 6A depicts a non-limiting example of a method of extracting VH and VK from the output pools from Round 3 and Round 4 of panning. Briefly, a Sanger forward primer and reverse CDRH3 primer was used to extract the VH portion and a CDRH3 forward and VK Sanger reverse primer was used to extract the VK fragment. FIG. 6B depicts another non-limiting example of a method of extracting VH and VK from the output pool from Round 3 and Round 4 of panning. Briefly, a VH forward primer and a JH reverse primer were used to extract the VH fragment. For the VK fragment, a FW3 forward primer and a JK reverse primer were used. FIG. 6C depicts log-log frequency plots generated from NGS results of clones that showed up across replicates. FIG. 6D depicts the distribution of hits (hashed curves were the non-selected clones; solid curves were the selected clones). FIG. 6E depicts the statistical properties of anti-CXCR5 antibodies which include CDRH3 length, Grand Average Hydropathy (GRAVY), bulkiness of CDRH3, aliphaticity, polarity, charge, basicity, acidicity, and aromaticity. FIG. 6F depicts the humanness of anti-CXCR5 antibodies. The percent amino acid identity was compared to the SHL germline amino acid sequences.

### FACS Screening

Round 3 and Round 4 panning outputs were plated for single colonies. Single colonies were picked, grown, and glycerol stocked in order to generate periplasmic extract (PPE) to test on the target cell line, parent cell line, and an off-target cell line. For PPE generation, cultures were grown overnight from the stocks of the selected scFv clones. Once the density reached the desired OD, IPTG was added to induce the expression of the clones. When cultures were ready, the plates were centrifuged to pellet cells and the supernatant was decanted. The pellets were treated with osmotic solution to release the periplasmic extracts. The cell debris was centrifuged at maximum speed for 10 minutes, and the PPE was collected and stored at −80° C. until further use. For the FACS screen, cells (desired target, parental, or off-target cell line) were plated at 100,000 cells per well and incubated with 25 μL of filtered periplasmic extract on ice for an hour. After an hour, cells were washed once with FACS buffer (1× PBS+0.5% BSA) and then incubated with a secondary anti-myc PE antibody for 30 minutes. The cells were then washed twice and resuspended in a final volume of 1% paraformaldehyde in 1× PBS and analyzed by FACS.

PPE was generated from single colonies and incubated on a positive cell line. FIG. 6G demonstrates that the scFvs were CXCR5 binders. FIG. 6H depicts representative FACS data. Positive scFv clones were reformatted into IgG1 and tested again on a CXCR5 positive cell line. The green shift in FIG. 6H indicates that the antibodies bound to CXCR5 and not to any off-target cell lines or to the parent cell line.

### Example 7. Functional Assays and Antibody Characterization

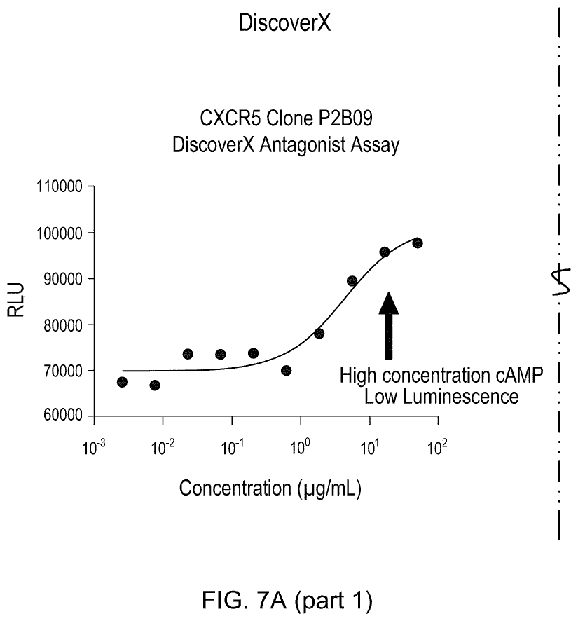

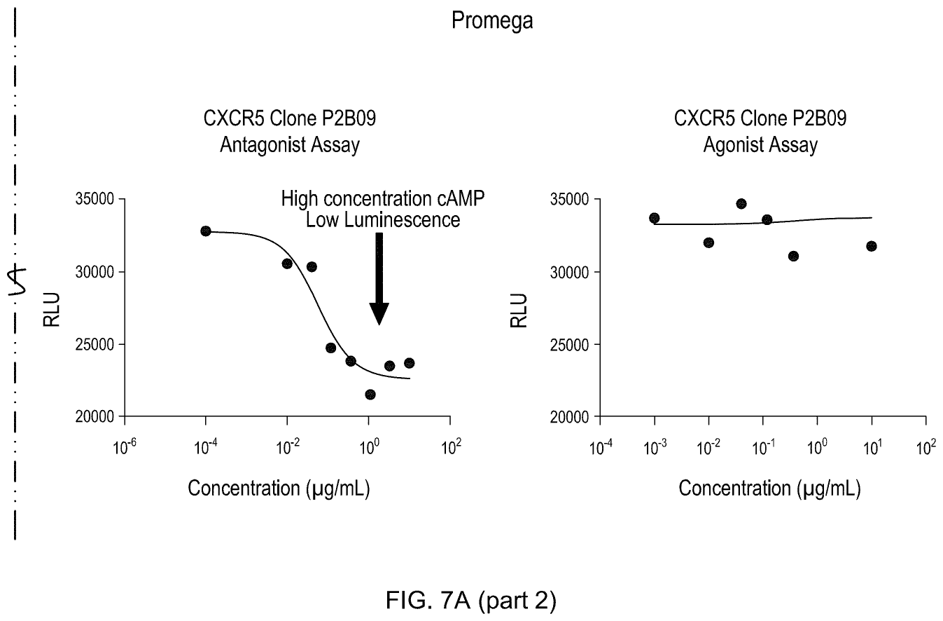

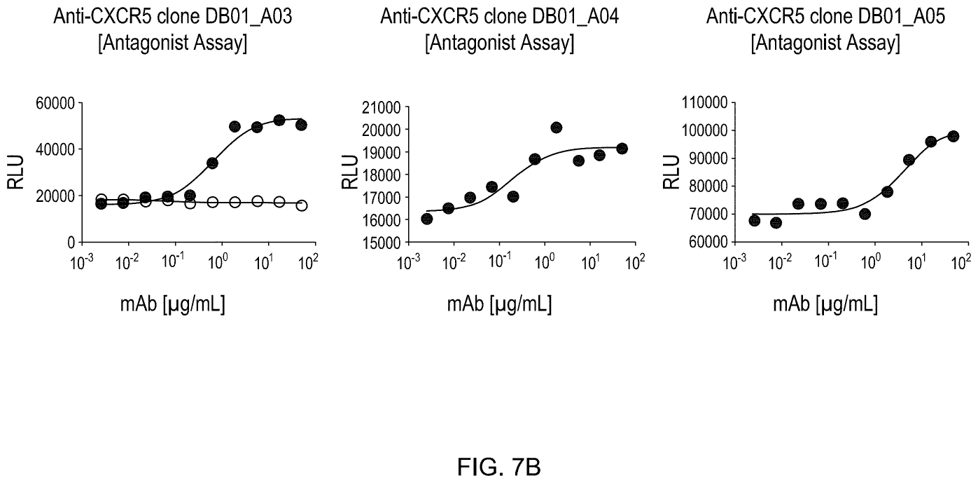

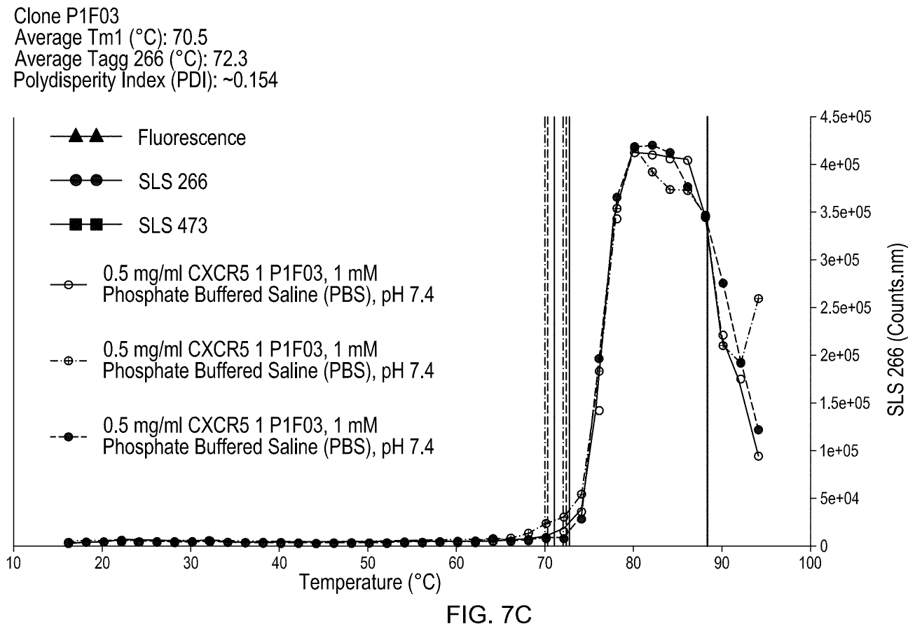

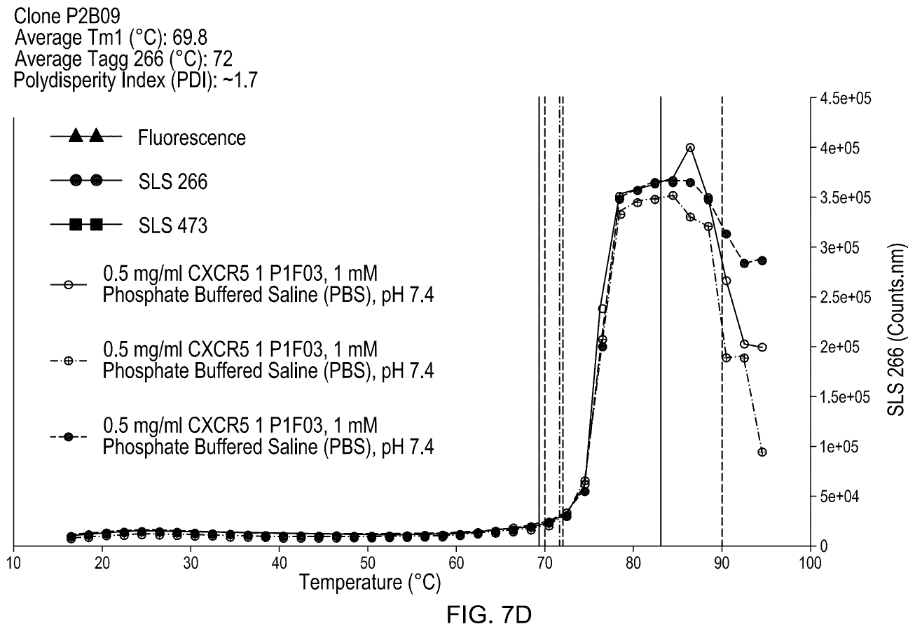

The functionality of selected CXCR5 antibodies was tested in both the DISCOVERX® cAMP Hunter eXpress GPCR assay, and the PROMEGA® cAMP-Glo Assay (both commercially available). FIG. 7A depicts representative data from this experiment. The luminescent signal was different for both assay formats. In the DISCOVERX® format, a higher concentration of cAMP was indicated by a high luminescence signal. For the PROMEGA® assay, a high concentration of cAMP levels was indicated by a low luminescence signal.

For the DISCOVERX® assay, cells were seeded in 100 μL of cell plating reagent and incubated overnight. The next day, the plating reagent was replaced with 30 μL of cell assay buffer. For the agonist testing mode, the cells were treated with 15 μL of 3× agonist prepared in cell assay buffer and 3× forskolin was included for G-stimulatory targets. Following treatment, the cells were incubated for 30 minutes at 37° C. and the cAMP Hunter eXpress GPCR detection protocol was followed. In antagonist mode, the cells were treated with 7.5 μL of 6× antagonist prepared in cell assay buffer and incubated for 15 minutes at 37° C. following incubation, the cells were treated with 7.5 μL 6× agonist prepared in cell assay buffer and 6× forskolin was included for G-stimulatory targets. Following treatment, the cells were incubated for 30 minutes at 37° C. and the cAMP Hunter eXpress GPCR detection protocol was followed. Results are depicted in FIG. 7B.

Uncle (Unchained Labs) was used to characterize the antibodies by loading 8.8 μL of each antibody sample in triplicate. The application Tm and Tagg with optional DLS gave the average melting temperature, aggregation temperature, and polydispersity index of each antibody sample. Results are depicted in FIG. 7C and FIG. 7D.

### Example 8. FACS Screening of Affinity Matured Target Antibodies

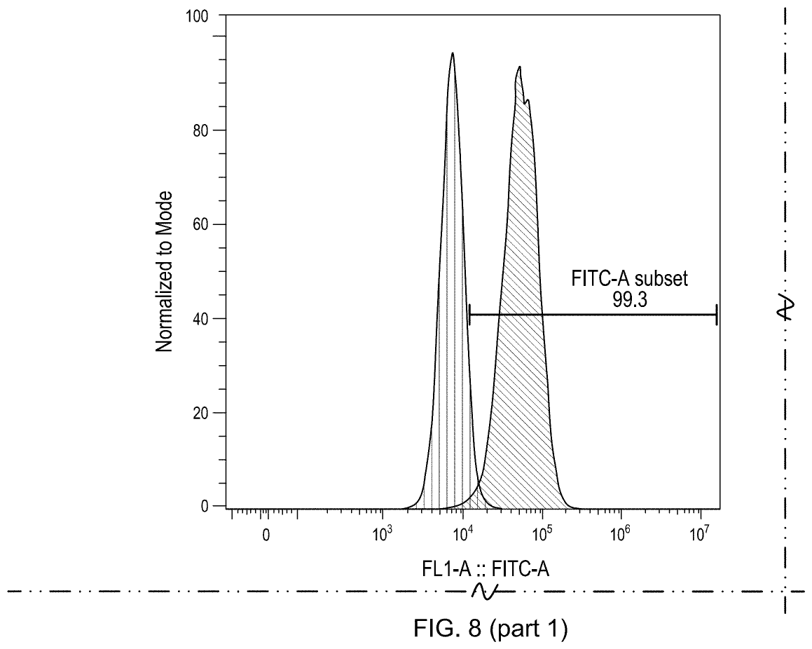

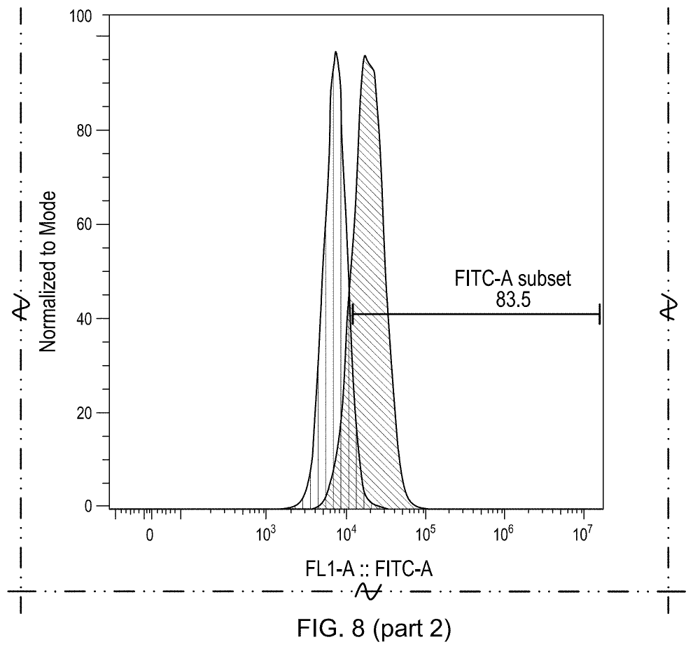

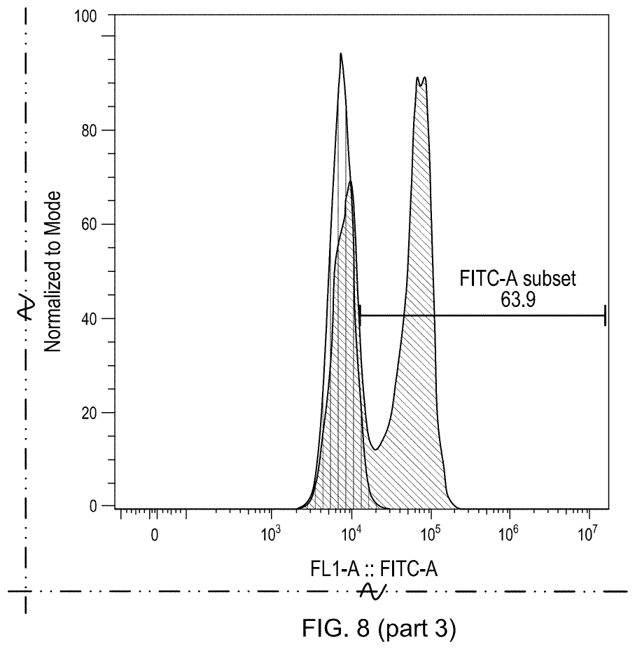

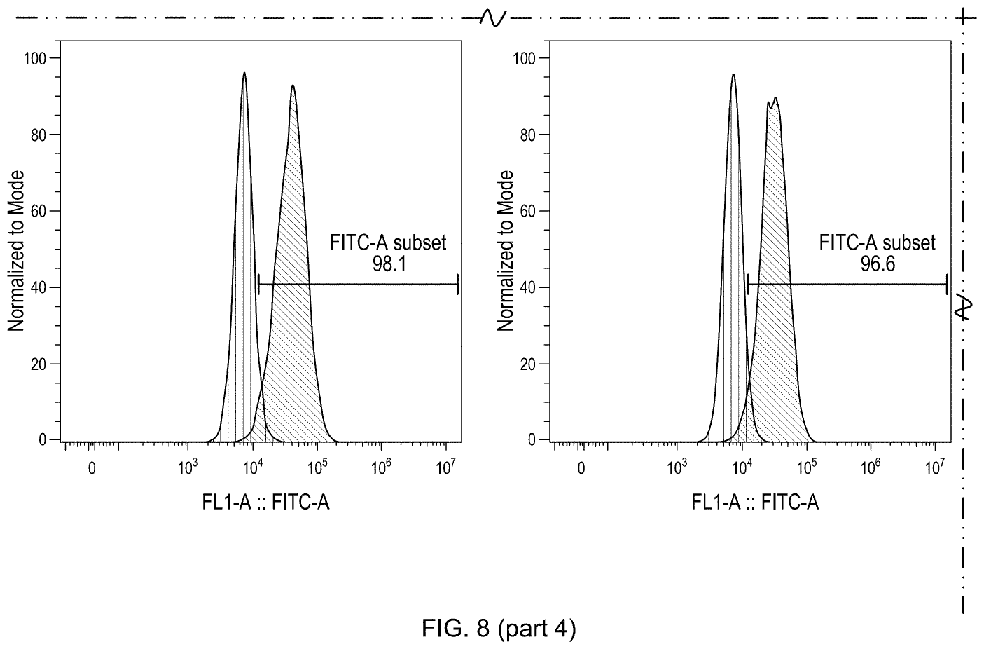

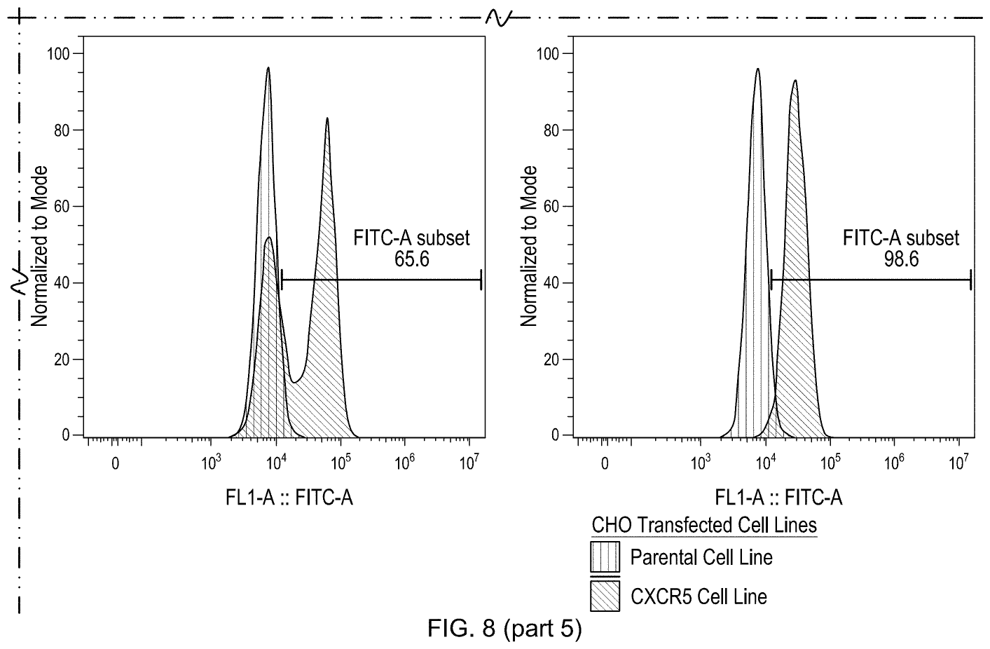

Briefly, cells (target cell line, parental cell line, or off-target cell line) were plated at 100,000 cells per well and incubated with 25 μL of filtered periplasmic extract on ice for an hour. After an hour, cells were washed once with FACS buffer (1× PBS+0.5% BSA) and then incubated with a secondary anti-myc FITC antibody for 30 minutes. The cells were then washed twice and resuspended in a final volume of 1% paraformaldehyde in 1× PBS and analyzed on FACS. FIG. 8 depicts data demonstrating binding of affinity matured target antibodies to CXCR5.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of selecting for a polypeptide that selectively binds to a target protein, said method comprising:
(a) contacting a first polypeptide pool comprising a plurality of polypeptides with a first entity that does not comprise said target protein to form a first mixture;
(b) removing said first entity from said first mixture, thereby generating a first depleted polypeptide pool;
(c) contacting said first depleted polypeptide pool with a second entity that comprises said target protein at its surface;
(d) collecting polypeptides that bind to said second entity, thereby generating a target polypeptide pool;
(e) contacting a second polypeptide pool comprising a plurality of polypeptides with the first entity to form a second mixture;
(f) removing the first entity from the second mixture, thereby generating a second depleted polypeptide pool;
(g) contacting said second depleted polypeptide pool with a third entity that does not comprise said target protein, wherein said third entity is the same or different from said first entity;
(h) collecting polypeptides that bind to said third entity, thereby generating an off-target polypeptide pool; and
(i) identifying at least one polypeptide that is present in said target polypeptide pool and is not present in said off-target polypeptide pool, thereby selecting for a polypeptide that selectively binds to said target protein.

2. The method of claim 1, further comprising, prior to (i), performing one or more rounds of (a)-(h), each successive round using a target polypeptide pool generated in (d) as a first polypeptide pool in (a), and using an off-target polypeptide pool generated in (h) as a second polypeptide pool in (e).

3. The method of claim 1, wherein said identifying of (i) comprises sequencing a polynucleotide tag attached to said at least one polypeptide.

4. (canceled)

5. The method of claim 1, wherein said identifying of (i) comprises sequencing polynucleotide tags attached to said plurality of target polypeptides and sequencing polynucleotide tags to said plurality of off-target polypeptides.

6. The method of claim 5, wherein each of said polynucleotide tags is different for each target polypeptide and for each off-target polypeptide.

7. (canceled)

8. The method of claim 2 wherein, for each successive round of (a)-(h), said first entity, said second entity, and said third entity are of a different type from a preceding round.

9. The method of claim 8, wherein, for each successive round of (a)-(h), said first entity, said second entity, and said third entity are from a different species than from a preceding round.

10. The method of claim 1, wherein said first entity comprises a plurality of first entities, said second entity comprises a plurality of second entities, and said third entity comprises a plurality of third entities.

11-14. (canceled)

15. The method of claim 1, wherein said target protein is a cell-surface protein, a membrane-bound protein, or a protein engineered to be expressed at a cell surface.

16. The method of claim 1, wherein said target protein is a transmembrane protein or an integral membrane protein

17. The method of claim 16, wherein said target protein is the transmembrane protein, and wherein said transmembrane protein is a multi-pass transmembrane protein.

18. The method of claim 1, wherein said target protein is selected from the group consisting of: a ligand-gated ion channel, a voltage-gated ion channel, and a G protein-coupled receptor (GPCR).

19-32. (canceled)

33. The method of claim 1, wherein any one of said first entity, said second entity, and said third entity comprises a whole cell.

34. (canceled)

35. (canceled)

36. The method of claim 1, wherein said first polypeptide pool, said second polypeptide pool, or both, is an antibody library comprising a plurality of antibodies or antibody fragments.

37. (canceled)

38. The method of claim 1 wherein said first polypeptide pool, said second polypeptide pool, or both comprises at least ten polypeptides.

39-46. (canceled)

47. The method of claim 1, further comprising, performing one or more affinity maturation steps on said at least one polypeptide to generate a polypeptide with increased affinity for said target protein.

48-73. (canceled)

74. The method of claim 3, wherein said sequencing comprises next generation sequencing.

75. The method of claim 33, wherein said first entity comprises a plurality of first cells, said second entity comprises a plurality of second cells, and said third entity comprises a plurality of third cells.

76. The method of claim 33, wherein, for each successive round of (a)-(h), the whole cell of said first entity, said second entity, and said third entity are different cells than a preceding round.
